SEQUENCE LISTING (37 sequences)

SEQ ID NO: 1 - DNA, 1335 bp, Artificial Sequence
  Antibody sequence
caggtgcagctggtgcagagcggcgctgaggtgaagaagcccggctcctccgtcaaggtcagctgcaaggcgagcgggcagacattcaccggatactacatgcattgggtgcgccaggccccggggcaggggctcgagtggatggggagaatcatccccatcctgggcatcgctcagaagttccagggacgcgtgaccatcaccgccgacaaatccaccagcaccgcctacatggaactgagctccctgcgctccgaggacaccgccgtgtattattgcgcccgcgggggcgacctgggcggcatggacgtgtggggccagggcacactagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcgcgggtgcaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 2 - protein, 445 aa, Artificial Sequence
  Antibody sequence
QVQLVQSGAEVKKPGSSVKVSCKASGQTFTGYYMHWVRQAPGQGLEWMGRIIPILGIAQKFQGRVTITADKSTSTAYMELSSLRSEDTAVYYCARGGDLGGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLAGAPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 657 aa, Artificial Sequence
  Antibody sequence
GAGATCGTGCTGACCCAGAGCCCCCTCTCGCTGCCCGTGACCCCCGGGGAGCCCGCCAGCATCTCCTGCCGCAGTAGCCAGAGCCTGAGGCATTCCAATGGCTACAACTACCTGGACTGGTACCTGCAGAAACCCGGCCAGAGCCCCCAGCTGCTCATCTACCTGGCGAGTAACCGCGCCAGCGGGGTGCCCGACCGCTTCAGCGGCTCCGGCAGTGGAACCGACTTCACCCTGAAGATCTCCCGCGTGGAGGCGGAGGACGTGGGGGTGTATTACTGTATGCAGGCCCTCCAGATCCCCCCCACGTTCGGCCCCGGCACCAAGGTGGACATCAAACGCACCGTCGCCGCCCCGACCGTGAGCATTTTCCCTCCCAGCTCCGAGCAGCTGACGTCCGGCGGCGCCTCTGTGGTGTGCTTCCTCAACAACTTCTACCCCAAGGACATCAACGTGAAGTGGAAGATCGACGGCTCCGAGAGACAGAACGGCGTGCTGAACAGCTGGACCGACCAGGACAGCAAGGACTCCACCTACAGTATGAGCTCCACCCTGACCCTGACCAAGGACGAGTACGAGAGGCATAACTCTTATACCTGCGAGGCGACCCATAAGACCAGCACCTCCCCCATCGTCAAGAGCTTCAACCGCAACGAATGC

SEQ ID NO: 4 - protein, 219 aa, Artificial Sequence
  Antibody sequence
EIVLTQSPLSLPVTPGEPASISCRSSQSLRHSNGYNYLDWYLQKPGQSPQLLIYLASNRASGVPDRFSGSGSGTDFTLKISRVEAEDVGVYYCMQALQIPPTFGPGTKVDIKRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 1353 bp, Artificial Sequence
  Antibody sequence
caggtccaattagtgcaatctgggtctgagttgaagaagcctggggcctcagtgaaggtttcctgcaaggcctctggatacaccttcactaactatggaatgaactgggtgcgacaggcccctggacaagggctcgagtggatgggatggataaacaccagaaatggaaagtcaacatatgttgatgacttcaaggggcggtttgtcttctccttggacacctctgtcagcacggcatatctacagatcagcagcctaaaggctgacgacactgcagtgtattactgtgcgagagaagggaatatggatggttacttcccttttacttactggggccagggtacactagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcgcgggtgcaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 6 - protein, 451 aa, Artificial Sequence
  Antibody sequence
QVQLVQSGSELKKPGASVKVSCKASGYTFTNYGMNWVRQAPGQGLEWMGWINTRNGKSTYVDDFKGRFVFSLDTSVSTAYLQISSLKADDTAVYYCAREGNMDGYFPFTYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLAGAPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - DNA, 642 bp, Artificial Sequence
  Antibody sequence
gatattgtcatgactcagtctccatcatccctgtccgcatcagtaggagacagggtcaccatcacctgcaaggcttctcagaatgtgggtactaatgtagcctggtatcaacagaaaccagggaaagctcctaaagcactgatttactcggcatcctatcggtacagtggagtccctgatcgcttctcaggcagtggatccgggacagatttcactctcaccatcagcagtctgcagcctgaagacttcgcaacgtattactgtcagcaatataacagctatcctctcacgttcggtggtggtaccaaggtggaaataaaacgtacggatgctgcaccgactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 8 - protein, 214 aa, Artificial Sequence
  Antibody sequence
DIVMTQSPSSLSASVGDRVTITCKASQNVGTNVAWYQQKPGKAPKALIYSASYRYSGVPDRFSGSGSGTDFTLTISSLQPEDFATYYCQQYNSYPLTFGGGTKVEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 40 aa, Heloderma horridum
HGEGTFTSDLSKQMEEEAVRLFIEWLKNGGPSSGAPPPSG

SEQ ID NO: 10 - protein, 30 aa, Homo Sapiens
HGEGTFTSDVSSYLEGQAAKEFIAWLVKGR

SEQ ID NO: 11 - DNA, 1494 bp, Artificial Sequence
  Antibody-Exendin fusion
catggggagggcactttcactagcgacctgagcaagcagatggaagaagaggccgtgaggctgttcattgagtggctcaagaacggaggcccctcctccggcgcccccccccctagcggcggatccggaggcgggggcagtggcgggggaggtagcggtcaggtgcagctggtgcagagcggcgctgaggtgaagaagcccggctcctccgtcaaggtcagctgcaaggcgagcgggcagacattcaccggatactacatgcattgggtgcgccaggccccggggcaggggctcgagtggatggggagaatcatccccatcctgggcatcgctcagaagttccagggacgcgtgaccatcaccgccgacaaatccaccagcaccgcctacatggaactgagctccctgcgctccgaggacaccgccgtgtattattgcgcccgcgggggcgacctgggcggcatggacgtgtggggccagggcacactagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcgcgggtgcaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 12 - protein, 498 aa, Artificial Sequence
  Antibody-Exendin fusion
HGEGTFTSDLSKQMEEEAVRLFIEWLKNGGPSSGAPPPSGGSGGGGSGGGGSGQVQLVQSGAEVKKPGSSVKVSCKASGQTFTGYYMHWVRQAPGQGLEWMGRIIPILGIAQKFQGRVTITADKSTSTAYMELSSLRSEDTAVYYCARGGDLGGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLAGAPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - DNA, 816 bp, Artificial Sequence
  Antibody-Exendin fusion
catggggagggcactttcactagcgacctgagcaagcagatggaagaagaggccgtgaggctgttcattgagtggctcaagaacggaggcccctcctccggcgcccccccccctagcggcggatccggaggcgggggcagtggcgggggaggtagcggtgagatcgtgctgacccagagccccctctcgctgcccgtgacccccggggagcccgccagcatctcctgccgcagtagccagagcctgaggcattccaatggctacaactacctggactggtacctgcagaaacccggccagagcccccagctgctcatctacctggcgagtaaccgcgccagcggggtgcccgaccgcttcagcggctccggcagtggaaccgacttcaccctgaagatctcccgcgtggaggcggaggacgtgggggtgtattactgtatgcaggccctccagatcccccccacgttcggccccggcaccaaggtggacatcaaacgcaccgtcgccgccccgaccgtgagcattttccctcccagctccgagcagctgacgtccggcggcgcctctgtggtgtgcttcctcaacaacttctaccccaaggacatcaacgtgaagtggaagatcgacggctccgagagacagaacggcgtgctgaacagctggaccgaccaggacagcaaggactccacctacagtatgagctccaccctgaccctgaccaaggacgagtacgagaggcataactcttatacctgcgaggcgacccataagaccagcacctcccccatcgtcaagagcttcaaccgcaacgaatgc

SEQ ID NO: 14 - protein, 272 aa, Artificial Sequence
  Antibody-Exendin fusion
HGEGTFTSDLSKQMEEEAVRLFIEWLKNGGPSSGAPPPSGGSGGGGSGGGGSGEIVLTQSPLSLPVTPGEPASISCRSSQSLRHSNGYNYLDWYLQKPGQSPQLLIYLASNRASGVPDRFSGSGSGTDFTLKISRVEAEDVGVYYCMQALQIPPTFGPGTKVDIKRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - DNA, 1443 bp, Artificial Sequence
  Antibody-GLP-1 fusion
catggggagggcaccttcacctccgacgtcagctcttacctcgagggccaagccgccaaggagtttatcgcctggctcgtgaaggggaggacagtcgcggcgcccagccaggtgcagctggtgcagagcggcgctgaggtgaagaagcccggctcctccgtcaaggtcagctgcaaggcgagcgggcagacattcaccggatactacatgcattgggtgcgccaggccccggggcaggggctcgagtggatggggagaatcatccccatcctgggcatcgctcagaagttccagggacgcgtgaccatcaccgccgacaaatccaccagcaccgcctacatggaactgagctccctgcgctccgaggacaccgccgtgtattattgcgcccgcgggggcgacctgggcggcatggacgtgtggggccagggcacactagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcgcgggtgcaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 16 - protein, 481 aa, Artificial Sequence
  Antibody-GLP-1 fusion
HGEGTFTSDVSSYLEGQAAKEFIAWLVKGRTVAAPSQVQLVQSGAEVKKPGSSVKVSCKASGQTFTGYYMHWVRQAPGQGLEWMGRIIPILGIAQKFQGRVTITADKSTSTAYMELSSLRSEDTAVYYCARGGDLGGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLAGAPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - DNA, 765 bp, Artificial Sequence
  Antibody-GLP-1 fusion
catggggagggcaccttcacctccgacgtcagctcttacctcgagggccaagccgccaaggagtttatcgcctggctcgtgaaggggaggacagtcgcggcgcccagcgagatcgtgctgacccagagccccctctcgctgcccgtgacccccggggagcccgccagcatctcctgccgcagtagccagagcctgaggcattccaatggctacaactacctggactggtacctgcagaaacccggccagagcccccagctgctcatctacctggcgagtaaccgcgccagcggggtgcccgaccgcttcagcggctccggcagtggaaccgacttcaccctgaagatctcccgcgtggaggcggaggacgtgggggtgtattactgtatgcaggccctccagatcccccccacgttcggccccggcaccaaggtggacatcaaacgcaccgtcgccgccccgaccgtgagcattttccctcccagctccgagcagctgacgtccggcggcgcctctgtggtgtgcttcctcaacaacttctaccccaaggacatcaacgtgaagtggaagatcgacggctccgagagacagaacggcgtgctgaacagctggaccgaccaggacagcaaggactccacctacagtatgagctccaccctgaccctgaccaaggacgagtacgagaggcataactcttatacctgcgaggcgacccataagaccagcacctcccccatcgtcaagagcttcaaccgcaacgaatgc

SEQ ID NO: 18 - protein, 255 aa, Artificial Sequence
  Antibody-GLP-1 fusion
HGEGTFTSDVSSYLEGQAAKEFIAWLVKGRTVAAPSEIVLTQSPLSLPVTPGEPASISCRSSQSLRHSNGYNYLDWYLQKPGQSPQLLIYLASNRASGVPDRFSGSGSGTDFTLKISRVEAEDVGVYYCMQALQIPPTFGPGTKVDIKRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - DNA, 1512 bp, Artificial Sequence
  Antibody-Exendin fusion
catggggagggcactttcactagcgacctgagcaagcagatggaagaagaggccgtgaggctgttcattgagtggctcaagaacggaggcccctcctccggcgcccccccccctagcggcggatccggaggcgggggcagtggcgggggaggtagcggtcaggtccaattagtgcaatctgggtctgagttgaagaagcctggggcctcagtgaaggtttcctgcaaggcctctggatacaccttcactaactatggaatgaactgggtgcgacaggcccctggacaagggctcgagtggatgggatggataaacaccagaaatggaaagtcaacatatgttgatgacttcaaggggcggtttgtcttctccttggacacctctgtcagcacggcatatctacagatcagcagcctaaaggctgacgacactgcagtgtattactgtgcgagagaagggaatatggatggttacttcccttttacttactggggccagggtacactagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggctccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcgcgggtgcaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 20 - protein, 504 aa, Artificial Sequence
  Antibody-Exendin fusion
HGEGTFTSDLSKQMEEEAVRLFIEWLKNGGPSSGAPPPSGGSGGGGSGGGGSGQVQLVQSGSELKKPGASVKVSCKASGYTFTNYGMNWVRQAPGQGLEWMGWINTRNGKSTYVDDFKGRFVFSLDTSVSTAYLQISSLKADDTAVYYCAREGNMDGYFPFTYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLAGAPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - DNA, 801 bp, Artificial Sequence
  Antibody-Exendin fusion
catggggagggcactttcactagcgacctgagcaagcagatggaagaagaggccgtgaggctgttcattgagtggctcaagaacggaggcccctcctccggcgcccccccccctagcggcggatccggaggcgggggcagtggcgggggaggtagcggtgatattgtcatgactcagtctccatcatccctgtccgcatcagtaggagacagggtcaccatcacctgcaaggcttctcagaatgtgggtactaatgtagcctggtatcaacagaaaccagggaaagctcctaaagcactgatttactcggcatcctatcggtacagtggagtccctgatcgcttctcaggcagtggctccgggacagatttcactctcaccatcagcagtctgcagcctgaagacttcgcaacgtattactgtcagcaatataacagctatcctctcacgttcggtggtggtaccaaggtggaaataaaacgtacggtcgccgccccgaccgtgagcattttccctcccagctccgagcagctgacgtccggcggcgcctctgtggtgtgcttcctcaacaacttctaccccaaggacatcaacgtgaagtggaagatcgacggctccgagagacagaacggcgtgctgaacagctggaccgaccaggacagcaaggactccacctacagtatgagctccaccctgaccctgaccaaggacgagtacgagaggcataactcttatacctgcgaggcgacccataagaccagcacctcccccatcgtcaagagcttcaaccgcaacgaatgc

SEQ ID NO: 22 - protein, 267 aa, Artificial Sequence
  Antibody-Exendin fusion
HGEGTFTSDLSKQMEEEAVRLFIEWLKNGGPSSGAPPPSGGSGGGGSGGGGSGDIVMTQSPSSLSASVGDRVTITCKASQNVGTNVAWYQQKPGKAPKALIYSASYRYSGVPDRFSGSGSGTDFTLTISSLQPEDFATYYCQQYNSYPLTFGGGTKVEIKRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - DNA, 642 bp, Artificial Sequence
  Antibody sequence
gatattgtcatgactcagtctccatcatccctgtccgcatcagtaggagacagggtcaccatcacctgcaaggcttctcagaatgtgggtactaatgtagcctggtatcaacagaaaccagggaaagctcctaaagcactgatttactcggcatcctatcggtacagtggagtccctgatcgcttctcaggcagtggatccgggacagatttcactctcaccatcagcagtctgcagcctgaagacttcgcaacgtattactgtcagcaatataacagctatcctctcacgttcggtggtggtaccaaggtggaaataaaacgtacggtggccgccccgaccgtgagcattttccctcccagctccgagcagctgacgtccggcggcgcctctgtggtgtgcttcctcaacaacttctaccccaaggacatcaacgtgaagtggaagatcgacggctccgagagacagaacggcgtgctgaacagctggaccgaccaggacagcaaggactccacctacagtatgagctccaccctgaccctgaccaaggacgagtacgagaggcataactcttatacctgcgaggcgacccataagaccagcacctcccccatcgtcaagagcttcaaccgcaacgaatgc

SEQ ID NO: 24 - protein, 214 aa, Artificial Sequence
  Antibody sequence
DIVMTQSPSSLSASVGDRVTITCKASQNVGTNVAWYQQKPGKAPKALIYSASYRYSGVPDRFSGSGSGTDFTLTISSLQPEDFATYYCQQYNSYPLTFGGGTKVEIKRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - DNA, 1482 bp, Artificial Sequence
  Antibody-GLP-1 fusion
catggggagggcaccttcacctccgacgtcagctcttacctcgagggccaagccgccaaggagtttatcgcctggctcgtgaaggggaggggatccggaggcgggggcagtggcgggggaggtagcggtcaggtccaattagtgcaatctgggtctgagttgaagaagcctggggcctcagtgaaggtttcctgcaaggcctctggatacaccttcactaactatggaatgaactgggtgcgacaggcccctggacaagggctcgagtggatgggatggataaacaccagaaatggaaagtcaacatatgttgatgacttcaaggggcggtttgtcttctccttggacacctctgtcagcacggcatatctacagatcagcagcctaaaggctgacgacactgcagtgtattactgtgcgagagaagggaatatggatggttacttcccttttacttactggggccagggtacactagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggctccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcgcgggtgcaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 26 - protein, 494 aa, Artificial Sequence
  Antibody-GLP-1 fusion
HGEGTFTSDVSSYLEGQAAKEFIAWLVKGRGSGGGGSGGGGSGQVQLVQSGSELKKPGASVKVSCKASGYTFTNYGMNWVRQAPGQGLEWMGWINTRNGKSTYVDDFKGRFVFSLDTSVSTAYLQISSLKADDTAVYYCAREGNMDGYFPFTYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLAGAPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - DNA, 771 bp, Artificial Sequence
  Antibody-GLP-1 fusion
catggggagggcaccttcacctccgacgtcagctcttacctcgagggccaagccgccaaggagtttatcgcctggctcgtgaaggggaggggatccggaggcgggggcagtggcgggggaggtagcggtgatattgtcatgactcagtctccatcatccctgtccgcatcagtaggagacagggtcaccatcacctgcaaggcttctcagaatgtgggtactaatgtagcctggtatcaacagaaaccagggaaagctcctaaagcactgatttactcggcatcctatcggtacagtggagtccctgatcgcttctcaggcagtggctccgggacagatttcactctcaccatcagcagtctgcagcctgaagacttcgcaacgtattactgtcagcaatataacagctatcctctcacgttcggtggtggtaccaaggtggaaataaaacgtacggtcgccgccccgaccgtgagcattttccctcccagctccgagcagctgacgtccggcggcgcctctgtggtgtgcttcctcaacaacttctaccccaaggacatcaacgtgaagtggaagatcgacggctccgagagacagaacggcgtgctgaacagctggaccgaccaggacagcaaggactccacctacagtatgagctccaccctgaccctgaccaaggacgagtacgagaggcataactcttatacctgcgaggcgacccataagaccagcacctcccccatcgtcaagagcttcaaccgcaacgaatgc

SEQ ID NO: 28 - protein, 257 aa, Artificial Sequence
  Antibody-GLP-1 fusion
HGEGTFTSDVSSYLEGQAAKEFIAWLVKGRGSGGGGSGGGGSGDIVMTQSPSSLSASVGDRVTITCKASQNVGTNVAWYQQKPGKAPKALIYSASYRYSGVPDRFSGSGSGTDFTLTISSLQPEDFATYYCQQYNSYPLTFGGGTKVEIKRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - DNA, 324 bp, Artificial Sequence
  Antibody sequence
caggtgcagctggtgcagagcggcgctgaggtgaagaagcccggctcctccgtcaaggtcagctgcaaggcgagcgggcagacattcaccggatactacatgcattgggtgcgccaggccccggggcaggggctcgagtggatggggagaatcatccccatcctgggcatcgctcagaagttccagggacgcgtgaccatcaccgccgacaaatccaccagcaccgcctacatggaactgagctccctgcgctccgaggacaccgccgtgtattattgcgcccgcgggggcgacctgggcggcatggacgtgtggggccagggc

SEQ ID NO: 30 - protein, 108 aa, Artificial Sequence
  Antibody sequence
QVQLVQSGAEVKKPGSSVKVSCKASGQTFTGYYMHWVRQAPGQGLEWMGRIIPILGIAQKFQGRVTITADKSTSTAYMELSSLRSEDTAVYYCARGGDLGGMDVWGQG

SEQ ID NO: 31 - DNA, 351 bp, Artificial Sequence
  Antibody sequence
gagatcgtgctgacccagagccccctctcgctgcccgtgacccccggggagcccgccagcatctcctgccgcagtagccagagcctgaggcattccaatggctacaactacctggactggtacctgcagaaacccggccagagcccccagctgctcatctacctggcgagtaaccgcgccagcggggtgcccgaccgcttcagcggctccggcagtggaaccgacttcaccctgaagatctcccgcgtggaggcggaggacgtgggggtgtattactgtatgcaggccctccagatcccccccacgttcggccccggcaccaaggtggacatcaaacgcaccgtcgccgcc

SEQ ID NO: 32 - protein, 117 aa, Artificial Sequence
  Antibody sequence
EIVLTQSPLSLPVTPGEPASISCRSSQSLRHSNGYNYLDWYLQKPGQSPQLLIYLASNRASGVPDRFSGSGSGTDFTLKISRVEAEDVGVYYCMQALQIPPTFGPGTKVDIKRTVAA

SEQ ID NO: 33 - protein, 19 aa, Artificial Sequence
  signal sequence
MGWSCIILFLVATATGVHS

SEQ ID NO: 34 - protein, 23 aa, Artificial Sequence
  linker
GSGGGGSGGGGSGGGGSGGGGSG

SEQ ID NO: 35 - protein, 6 aa, Artificial Sequence
  linker
TVAAPS

SEQ ID NO: 36 - protein, 7 aa, Artificial Sequence
  linker
ASTKGPS

SEQ ID NO: 37 - protein, 5 aa, Artificial Sequence
  linker
GSSSS

(19) United States
(12) Patent Application Publication
(10) Pub. No.: US 2012/0058116 A1
(43) Pub. Date: Mar. 8, 2012

(54) FGFR1C ANTIBODY COMBINATIONS

(76) Inventors: Andrew Beaton, Hertfordshire (GB); Sean Matthew Cleveland, Hertfordshire (GB); Gerald Wayne Gough, Hertfordshire (GB); Mark Andrew Paulik, Durham, NC (US)

(21) Appl. No.: 13/265,887

(22) PCT Filed: Apr. 22, 2010
(86) PCT No.: PCT/EP2010/055320

§ 371 Date: Oct. 24, 2011

Related U.S. Application Data
(60) Provisional application No. 61/172,386, filed on Apr. 24, 2009.

(51) Int. Cl. A61K 39/395; A61P 1/00; A61P 3/00; C07K 19/00; A61P 3/04

(52) U.S. Cl. 424/134.1; 530/387.3; 424/158.1

(57) ABSTRACT

The invention relates to combinations of FGFR1c antagonists with agonist peptides and provide dual targeting proteins which bind to FEFR1c comprising an antigen binding protein which is capable of binding to FGFR1c and which is linked to one or more agonist peptides, methods of making such constructs and uses thereof, particularly in treating obesity.

### BACKGROUND

Fibroblast Growth Factor Receptors (FGFRs) 1-5 have common structural features which consist of an extracellular ligand-binding section composed of three domains (Ig domains I, II, and III), a transmembrane domain, and an intracellular tyrosine kinase catalytic domain. At least 22 ligands (FGFs) are known that signal through FGFRs 1-5. In FGFR-1 alternative splicing of the exon encoding the third IgG-like domain produces the b- or c-splice form both of which have distinct ligand-binding preferences. The FGFR1c splice form has been shown to regulate food intake (see Experimental Neurology 137, 318-323 (1996) and Am J Physiol Endocrinol Metab 292, 964-976 (2007)).

Some of the energy balance regulating hormones secreted by the gastrointestinal tract (GI) have been implicated as possible therapeutic agents for the treatment of obesity (see Drugs 2008; 68 (2) 147-163)). These include glucagon like peptide-1 (GLP-1), as well as fragments, variants, and/or conjugates thereof. GLP-1 is an incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of β-cells.

Native GLP-1 has a very short serum half-life (<5 minutes). Accordingly, it is not currently feasible to exogenously administer native GLP-1 as a therapeutic treatment.

### SUMMARY OF INVENTION

The present invention relates to the combination of an FGFR1c antagonist, for example an FGFR1c antibody, with an agonist peptide, for example a GLP-1 agonist molecule. The present invention further relates to the use of this combination in therapy, in particular for use in treating obesity, diabetes, metabolic syndrome and related diseases. The present invention provides a method for reducing body weight comprising administration of an anti-FGFR1c antagonist, for example an FGFR1c antibody, with an agonist peptide, for example a GLP-1 agonist molecule.

The present invention also provides a dual targeting protein comprising an FGFR1c antibody which is linked to one or more agonist peptides, for example a GLP1 agonist molecule, for example GLP-1 or exendin-4.

The invention also provides a polynucleotide sequence encoding a heavy chain of any of the dual targeting proteins described herein, and a polynucleotide encoding a light chain of any of the dual targeting proteins described herein. Such polynucleotides represent the coding sequence which corresponds to the equivalent polypeptide sequences, however it will be understood that such polynucleotide sequences could be cloned into an expression vector along with a start codon, an appropriate signal sequence and a stop codon.

The invention also provides a recombinant transformed or transfected host cell comprising one or more polynucleotides encoding a heavy chain and a light chain of any of the dual targeting proteins described herein.

The invention further provides a method for the production of any of the dual targeting proteins described herein which method comprises the step of culturing a host cell comprising a first and second vector, said first vector comprising a polynucleotide encoding a heavy chain of any of the dual targeting proteins described herein and said second vector comprising a polynucleotide encoding a light chain of any of the dual targeting proteins described herein, in a suitable culture media, for example serum-free culture media.

The invention further provides a pharmaceutical composition comprising a dual targeting protein as described herein and a pharmaceutically acceptable carrier.

### DEFINITIONS

“Agonist Peptide” as used herein means any energy regulating hormone secreted from any endocrine/neuroendocrine organ. These include but are not limited to GLP-1 agonist molecules including GLP-1 and exendin molecules. As used herein “agonist peptides” also include, but are not limited to Adiponectin, Adrenomodulin, Adropin, Apelin, Amylin, Bombesin, Calcitonin and Calcitonin gene related peptide (CGRP), Cocaine- and amphetamine-regulated transcript (CART), Cholecystokinin (CCK), Des-acyl-ghrelin, Enterostatin, Endothelin, Galanin-like peptide(GALP), Gastrin-releasing peptide(GRP), Glicentin, glucagon, Glucose-dependent insulinotropic peptide (GIP), insulin, intermedin, leptin, motilin, Melanocortin agonist peptide (MTII), Neuromedin B, Neurotensin, Neuromedin U, Obestatin, Orexin A, Orexin B, oxyntomodulin, oxytocin, pituatary adenylate cyclase activating polypeptide (PACAP-38), PP, PYY (PYY3-36 and PYY13-36), Peptide W, secretin, stresscopin, Thyrotropin-releasing hormone (TRH), Urocortin, VIP and Xenin.

“GLP-1 agonist molecule” as used herein means any molecule capable of agonising the GLP-1 Receptor. These include but are not limited to, any polypeptide which has at least one GLP-1 activity, including GLP-1, Exendin 3, Exendin-4, oxyntomodulin, and including any analogues, fragments and/or variants and/or conjugates thereof, for example GLP-1(7-37).

The term “antigen binding protein” as used herein refers to antibodies, antibody fragments, for example a domain antibody (dAb), ScFv, FAb, FAb₂, and other protein constructs which are capable of binding to FGFR1c. Antigen binding molecules may comprise at least one Ig variable domain, for example antibodies, domain antibodies, Fab, Fab', F(ab')2, Fv, ScFv, diabodies, mAbdAbs, affibodies, heteroconjugate antibodies or bispecifics. In one embodiment the antigen binding molecule is an antibody. In another embodiment the antigen binding molecule is a dAb, i.e. an immunoglobulin single variable domain such as a VH, VHH or VL that specifically binds an antigen or epitope independently of a different V region or domain. Antigen binding molecules may be a combination of antibodies and antigen binding fragments such as for example, one or more domain antibodies and/or one or more ScFvs linked to a monoclonal antibody. Antigen binding molecules may also comprise a non-Ig domain for example a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (transbody); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to FGFR1c. As used herein “antigen binding protein” will be capable of antagonising and/or neutralising human FGFR1c. In addition, an antigen binding protein may block FGFR1c activity by binding to FGFR1c and preventing a natural ligand from binding and/or activating the receptor.

As used herein “FGFR1c antagonist” includes any compound capable of reducing and or eliminating at least one activity of FGFR1c. By way of example, an FGFR1c antagonist may bind to FGFR1c and that binding may directly reduce or eliminate FGFR1c activity or it may work indirectly by blocking at least one ligand from binding the receptor.

As used herein “protein scaffold” includes but is not limited to an Ig scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. Such protein scaffolds may comprise antigen-binding sites in addition to the one or more constant regions, for example where the protein scaffold comprises a full IgG. Such protein scaffolds will be capable of being linked to other protein domains, for example agonist peptides.

### DETAILED DESCRIPTION OF INVENTION

The present invention provides compositions comprising an FGFR1c antagonist and an agonist peptide, for example a GLP-1 agonist molecule. The present invention also provides the combination of an FGFR1c antagonist and an agonist peptide, for example a GLP-1 agonist molecule, for use in therapy. The present invention also provides a method of treating obesity, diabetes, metabolic syndrome and related diseases by administering an FGFR1c antagonist in combination with an agonist peptide. The present invention also provides a method of reducing body weight by administering an FGFR1c antagonist in combination with an agonist peptide for example a GLP-1 agonist molecule. The FGFR1c antagonist and the agonist peptide may be administered separately, sequentially or simultaneously.

Such FGFR1c antagonists may be antigen binding proteins such as FGFR1c antibodies or soluble receptors such as FGFR1c-Fc (e.g. FP-1039 in development by FivePrime™) or they may be small molecule antagonists such as PD166866 (Panek et al. J, Pharmacol. Exp. Ther. 286, 569-577 (1998)).

The antigen binding protein of the present invention may comprise an Ig scaffold, for example an IgG scaffold or IgA scaffold. The IgG scaffold may comprise all the domains of an antibody (i.e. CH1, CH2, CH3, VH, VL). The dual targeting protein of the present invention may comprise an IgG scaffold selected from IgG1, IgG2, IgG3, IgG4 or IgG4PE.

In one embodiment, agonist peptides of use in the present invention may be selected from GLP-1 agonist molecules, Adiponectin, Adrenomodulin, Adropin, Apelin, Amylin, Bombesin, Calcitonin and Calcitonin gene related peptide (CGRP), Cocaine- and amphetamine-regulated transcript (CART), Cholecystokinin (CCK), Des-acyl-ghrelin, Enterostatin, Endothelin, Galanin-like peptide (GALP), Gastrin-releasing peptide (GRP), Glicentin, Glucagon, insulin, intermedin, leptin, motilin, Melanocortin agonist peptide (MTII), Neuromedin B, Neurotensin, Neuromedin U, Obestatin, Orexin A and B, oxyntomodulin, oxytocin, pituatary adenylate cyclase activating polypeptide (PACAP-38), PP, PYY (PYY3-36 and PYY13-36), Peptide W, secretin, stresscopin, Thyrotropin-releasing hormone (TRH), Urocortin, VIP and Xenin.

Glucagon-Like peptide 1 (GLP-1); GLP-1 is an incretin hormone which potentiates post-prandial insulin release. GLP-1 also inhibits glucagon secretion, delays gastric emptying and inhibits food intake in animals and humans. For further details see Field et al., Drugs 2008; 68 (2) 147-163.

Amlyin: Amylin is a 37 amino acid peptide hormone that is co-secreted with insulin in response to food intake. Exogenous amylin potently reduces food intake in humans and rodents, slows gastric emptying and reduces postprandial glucagons secretion. For further details see Field et al., Drugs 2008; 68 (2) 147-163.

Neuromedim U (NMU): NMU is a 25 amino acid peptide expressed in the upper GI tract and shares limited homology with other GI peptides such s VIP and PP. NMU reduces gastric acid secretion and stomach emptying.

Cholecystokinin (CCK): CCK was the first gut hormone to be demonstrated to reduce food intake. Bioactive CCK is derived from pro-CCK and consists of a mixture of several cleavage products fo varying lengths, each of which includes the minimal epitope for bioactivity, a carboxy-terminal-amidated, tyrosyl O-sulphated heptapeptide. For further details see Field et al., Drugs 2008; 68 (2) 147-163.

Peptide YY (PYY): PYY is a PP-fold peptide hormone with the predominant circulating form being PYY₃₋₃₆. PYY is relased by endocrine L-cells in the GI mucosa in response to food intake. Several studies have shown the ability of long-term PYY₃₋₃₆ administration to cause weight loss in animal models of obesity. For further details see Field et al., Drugs 2008; 68 (2) 147-163.

Pancreatic Polypeptide (PP): PP is a 36 amino acid peptide principally secreted by pancreatic islet cells but is also expressed in the distal gut. Intraperitoneal administration of PP reduces food intake, gastric emptying, gastric ghrelin mRNA expression, bodyweight gain and insulin resistance in animal models. For further details see Field et al., Drugs 2008; 68 (2) 147-163.

Enterostatin: Enterostatin is a pentapeptide which decreases food intake whether given peripherally or centrally and has been reported to selectively decrease fat intake. For further details see Nogueiras et al., Drug Discovery Today: Disease Mechanisms, 3: 463-470 (2006)).

Leptin: Human leptin I 167 amino acids in length and predominantly secreted by adipocytes and the stomach. Peripheral administration off leptin to ob/ob mice reduces food intake and restores normal body weight.

In one embodiment the agonist peptide is a GLP-1 agonist molecule.

In one embodiment the FGFR1c antagonist is an antigen binding protein and the agonist peptide is a GLP-1 agonist molecule. In one such embodiment the antigen binding protein is an FGFR1c antibody.

The FGFR1c antagonist and the agonist peptide, for example the GLP-1 agonist molecule, may be administered as a mixture of separate molecules which are administered at the same time i.e. co-administered, or are administered within 24 hours of each other, for example within 20 hours, or within 15 hours or within 12 hours, or within 10 hours, or within 8 hours, or within 6 hours, or within 4 hours, or within 2 hours, or within 1 hour, or within 30 minutes of each other. The agonist peptide may be administered more frequently than the FGFR1c antagonist, for example the FGFR1c antagonist may be dosed once a week, once every two weeks, once a month, once every 2 months, or once every 3 months. The agonist peptide may be dosed daily, every other day, twice a week, once a week, once every two weeks, once a month, or once every 2 months.

Any of the agonist peptides of the invention may be linked to an IgG or albumin or other suitable half life extenders. Combinations of the invention include combinations of an FGFR1c antagonist and an agonist peptide wherein the agonist peptide is fused to another molecule to extend its half-life, for example a protein scaffold, e.g. an IgG scaffold, for example an isolated antibody Fc region or an intact antibody, or human serum albumin. Examples of such half-life extended GLP-1 agonist molecules which are GLP-1 agonist molecules of use in the present invention include human serum albumin fusions such as Albiglutide (Syncria™) (Diabetes 2004, 53, 2492-2500). Other longer-acting forms of GLP-1 agonist molecules include GLP-1 linked Albudabs™ (Further details can be found in WO 03/002609, WO 2004/003019, WO 2004/058821, WO 2005/118642, WO 2006/059106 and WO 2008/096158) or derivatised versions of GLP-1 such as those described in J Med Chem 2000, 43, 1664-1669, for example Liraglutide.

In a further embodiment the antagonist and agonist are present as one molecule capable of interacting with two or more targets, for example the invention provides a dual targeting protein which is capable of antagonising FGFR1c and agonising a peptide receptor involved in regulating food intake, for example the invention provides a dual targeting protein which is capable of antagonising FGFR1c and agonising the GLP-1 Receptor.

In one embodiment the present invention provides a dual targeting protein comprising an antigen binding protein linked to one or more agonist peptides wherein the dual targeting protein is capable of binding FGFR1c and is also capable of agonising peptide receptor.

Such dual targeting proteins may comprise an antigen binding protein, for example a monoclonal antibody, which is linked to one or more agonist peptides. The invention provides methods of producing such dual targeting proteins and uses thereof, particularly uses in therapy.

Some examples of dual targeting proteins according to the invention, where an agonist peptide is linked to the N terminus of the light and/or heavy chains of an FGFR1c antagonist mAb, are set out in FIG. 8.

The compositions and dual targeting proteins of the present invention are capable of neutralising FGFR1c.

The term “neutralises” and grammatical variations thereof as used throughout the present specification in relation to dual targeting proteins and compositions of the invention means that a biological activity of the target is reduced, either totally or partially, in the presence of the dual targeting proteins of the present invention in comparison to the activity of the target in the absence of such dual targeting proteins. Neutralisation may be due to but not limited to one or more of blocking ligand binding, preventing the ligand activating the receptor, down regulating the receptor or affecting effector functionality.

Levels of neutralisation can be measured in several ways, for example in a receptor binding assay which may be carried out for example as described in Example 3. The neutralisation of FGFR1c in this assay is measured by assessing the decreased binding between the ligand and its receptor in the presence of neutralising dual targeting molecules or combinations of the present invention.

Other methods of assessing neutralisation are known in the art, and include, for example, Biacore™ assays to assess the decreased binding between the ligand and its receptor in the presence of neutralising dual targeting protein.

The FGFR1c antagonists of the present invention may also be capable of antagonising FGFR4.

In a further aspect of the present invention there is provided dual targeting proteins which have at least substantially equivalent neutralising activity to the dual targeting proteins exemplified herein.

Examples of such dual targeting proteins include FGFR1c antibodies which have a GLP-1 agonist molecule attached to the N-terminus of the heavy chain or the N-terminus of the light chain, Examples include a dual targeting protein comprising the VH sequence set out in SEQ ID NO:30 and the VL sequence set out in SEQ ID NO:32 wherein one or both of the Heavy and Light chain further comprise one or more GLP-1 agonist molecules linked to their N-terminus, for example the Exendin 4 set out in SEQ ID NO: 9 and/or the GLP-1 set out in SEQ ID NO: 10.

In one embodiment the present invention provides a dual targeting protein comprising an anti-FGFR1c antibody or antigen binding fragment thereof linked to a GLP-1 agonist molecule, wherein the anti-FGFR1c antibody or antigen binding fragment thereof comprises the the CDRs of the antibody set out in SEQ ID NO 2 and 4.

Other examples of such suitable antigen binding proteins of use in the present invention include FGFR1c antibodies such as those selected from any of the FGFR1c antibody sequences set out in WO2005037235, in particular the antibody which is described as FRI-A1 i.e. the VH and VL regions described in SEQ ID NO:15 and 16 of WO2005037235 or any antibody or antigen binding fragment thereof which comprises the CDRs of the FR1-A1 antibody, for example the CDRs set out in SEQ ID NO:9-14 of WO2005037235.

The CDR sequences of such antibodies can be determined by the Kabat numbering system (Kabat et al; Sequences of proteins of Immunological Interest NIH, 1987), the Chothia numbering system (Al-Lazikani et al., (1997) JMB 273,927-948), the contact definition method (MacCallum R. M., and Martin A. C. R. and Thornton J. M, (1996), Journal of Molecular Biology, 262 (5), 732-745) or any other established method for numbering the residues in an antibody and determining CDRs known to the skilled man in the art.

Other examples of such dual targeting proteins include anti-FGFR1c antibodies which have one or more agonist peptide molecules attached to the c-terminus or the n-terminus of the heavy chain or the c-terminus or n-terminus of the light chain.

Such dual targeting proteins may also have one or more further agonist peptides attached to the C-terminus and/or the N-terminus of the heavy chain and/or the C-terminus and/or N-terminus of the light chain. For example a dual targeting protein of the present invention may comprise an FGFR1c antibody with two or more agonist peptides attached to the N-terminus of each of the heavy chains, it may also comprise an FGFR1c antibody with two or more agonist peptides attached to the N-terminus of each of the light chains. One such dual targeting protein may be an FRFR1c antibody with two GLP-1 agonist molecules attached to the N-terminus of each heavy chain, wherein the C-terminus of the first GLP-1 agonist molecule is linked to the N-terminus of the heavy chain, and the c-terminus of the second GLP-1 agonist molecule is linked to the N-terminus of the first GLP-1 agonist molecule.

Antigen binding proteins of the present invention may be linked to agonist peptides by chemical conjugation or by genetic fusion. Chemical conjugation can be carried out by any suitable process which will be known to the skilled person in the art, for example using maleimide conjugation. Antigen binding proteins may be linked to agonist peptides by the the use of linkers. Examples of suitable linkers include peptide linkers, for example linkers comprising amino acid sequences which may be from 1 amino acid to 150 amino acids in length, or from 1 amino acid to 140 amino acids, for example, from 1 amino acid to 130 amino acids, or from 1 to 120 amino acids, or from 1 to 80 amino acids, or from 1 to 50 amino acids, or from 1 to 20 amino acids, or from 1 to 10 amino acids, or from 5 to 18 amino acids. Such sequences may have their own tertiary structure, for example, a linker of the present invention may comprise a single variable domain. The size of a linker in one embodiment is equivalent to a single variable domain. Suitable linkers may be of a size from 1 to 20 angstroms, for example less than 15 angstroms, or less than 10 angstroms, or less than 5 angstroms.

In one embodiment of the present invention at least one of the agonist peptides is linked to the antigen binding protein with a linker comprising from 1 to 150 amino acids, for example 1 to 20 amino acids, for example 1 to 10 amino acids. Such linkers may be selected from any one of those set out in SEQ ID NO 34-37, for example the linker may be ‘TVAAPS’, or the linker may comprise ‘GGGGS or between 1 and 6 repeats of the sequence ‘GGGGS’, or between 1 and 4 repeats of the sequence ‘GGGGS’, for example the linker may be ‘GGGGSGGGGS’, or ‘GGGGSGGGGSGGGGS’, or ‘GGGGSGGGGSGGGGSGGGGS’. Linkers of use in the dual targeting proteins of the present invention may comprise alone or in addition to other linkers, one or more sets of GS residues, for example ‘GSTVAAPS’ or ‘TVAAPSGS’ or ‘GSTVAAPSGS’. In another embodiment there is no linker between the agonist peptides, for example the between the GLP-1 agonist molecule and the antigen binding protein. In another embodiment the agonist peptide, for example the GLP-1 agonist molecule, is linked to the antigen binding protein by the linker ‘TVAAPS’. In another embodiment the agonist peptide, for example the GLP-1 agonist molecule, is linked to the antigen binding protein by the linker ‘VAAPSGS’. In another embodiment the agonist peptide, for example the GLP-1 agonist molecule, is linked to the antigen binding protein by the linker ‘GS’. In another embodiment the agonist peptide, for example the GLP-1 agonist molecule, is linked to the antigen binding protein by the linker ‘ASTKGPS’.

In another embodiment the agonist peptide, for example the GLP-1 agonist molecule, is directly linked to the antigen binding protein as a genetic fusion without the use of any additional linking sequence.

In one embodiment of the present invention there is provided a dual targeting protein according to the invention described herein and comprising a constant region such that the antibody has reduced ADCC and/or complement activation or effector functionality. In one such embodiment the heavy chain constant region may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering).

Antigen binding proteins of use in the present invention include full monoclonal antibodies comprising all the domains of an antibody, or antigen binding proteins of the present invention may comprise a non-conventional antibody structure, such as a monovalent antibody. Such monovalent antibodies may comprise a paired heavy and light chain wherein the hinge region of the heavy chain is modified so that the heavy chain does not homodimerise, such as the monovalent antibody described in WO2007059782. Other monovalent antibodies may comprise a paired heavy and light chain which dimerises with a second heavy chain which is lacking a functional variable region and CH1 region, wherein the first and second heavy chains are modified so that they will form heterodimers rather than homodimers, resulting in a monovalent antibody with two heavy chains and one light chain such as the monovalent antibody described in WO2006015371. Such monovalent antibodies can provide the antigen binding protein of the present invention to which agonist peptides can be linked.

Agonist peptides can be linked to the antigen binding protein at one or more positions. These positions include the C-terminus and the N-terminus of the antigen binding protein, for example at the C-terminus of the heavy chain and/or the C-terminus of the light chain of an antibody, or for example the N-terminus of the heavy chain and/or the N-terminus of the light chain of an antibody.

In one embodiment, a first agonist peptide is linked to the antigen binding protein and a second agonist peptide is linked to the first agonist peptide, for example where the antigen binding protein is a monoclonal antibody, a first agonist peptide may be linked to the c-terminus of the heavy chain of the antibody, and that epitope binding domain can be linked at its c-terminus to a second agonist peptide, or for example a first agonist peptide may be linked to the c-terminus of the light chain of the antibody and that first agonist peptide may be further linked at its c-terminus to a second agonist peptide, or for example a first agonist peptide may be linked to the n-terminus of the light chain of the antibody, and that first agonist peptide may be further linked at its n-terminus to a second agonist peptide, or for example a first agonist peptide may be linked to the n-terminus of the heavy chain of the antibody, and that first agonist peptide may be further linked at its n-terminus to a second agonist peptide.

Some agonist peptides may be suited to being linked to particular positions on the antigen binding protein, for example GLP-1 and Exendin 4 require a free N-terminus for maximum binding to their receptor, therefore GLP-1 and Exendin-4 are preferably linked via their C-terminus to the N-terminus of the antigen binding protein; PYY may require a free C-terminus for maximum binding to its receptor, therefore PYY is preferably linked via its N-terminus to the C-terminus of the antigen binding protein.

The invention also provides such compositions and dual targeting proteins for use in medicine, for example for use in the manufacture of a medicament for treating obesity, diabetes, metabolic syndrome and related diseases.

The compositions and dual targeting proteins of the present invention may be useful in the treatment of hyperglycemia, impaired glucose tolerance, beta cell deficiency, type 1 diabetes, type 2 diabetes, gestational diabetes, obesity or diseases characterised by overeating, insulin resistance, insulin deficiency, hyperinsulinemia, dyslipidemia, hyperlipidemia, hyperketonemia, hypertension, coronary artery disease, atherosclerosis, renal failure, neuropathy (e.g. autonomic neuropathy, parasympathetic neuropathy, and polyneuropathy), retinopathy, cataracts, metabolic disorders (e.g. insulin and/or glucose metabolic disorders), endocrine disorders, liver disorders (e.g. liver disease, cirrhosis of the liver, and disorders associated with liver transplant), and conditions associated with these diseases or disorders.

The invention provides a method of treating a patient suffering from one or more of the following diseases hyperglycemia, impaired glucose tolerance, beta cell deficiency, type 1 diabetes, type 2 diabetes, gestational diabetes, obesity or diseases characterised by overeating, insulin resistance, insulin deficiency, hyperinsulinemia, dyslipidemia, hyperlipidemia, hyperketonemia, hypertension, coronary artery disease, atherosclerosis, renal failure, neuropathy (e.g. autonomic neuropathy, parasympathetic neuropathy, and polyneuropathy), retinopathy, cataracts, metabolic disorders (e.g. insulin and/or glucose metabolic disorders), endocrine disorders, liver disorders (e.g. liver disease, cirrhosis of the liver, and disorders associated with liver transplant), and conditions associated with these diseases or disorders, comprising administering a therapeutic amount of a dual targeting protein of the invention.

In particular the compositions and dual targeting protein of the present invention may be useful in the treatment of obesity. The invention provides a method of treating a patient suffering from obesity comprising administering a therapeutic amount of a dual targeting protein of the invention.

In one embodiment the compositions or dual targeting proteins of the present invention can be used in the reduction of body weight in a patient.

In another embodiment the compositions or dual targeting proteins of the present invention can be used to reduce food intake in a patient.

In yet another embodiment the compositions or dual targeting proteins of the present invention can be used to inhibit gastric emptying in a patient.

The antigen binding proteins and dual targeting proteins of the present invention may be produced by transfection of a host cell with an expression vector comprising the coding sequence for the dual targeting protein of the invention. An expression vector or recombinant plasmid is produced by placing these coding sequences for the dual targeting protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary dual targeting protein light or heavy chain. In certain embodiments this second expression vector is identical to the first except insofar as the coding sequences and selectable markers are concerned, so to ensure as far as possible that each polypeptide chain is functionally expressed.

Alternatively, the heavy and light chain coding sequences for the dual targeting protein may reside on a single vector, for example in two expression cassettes in the same vector.

A selected host cell is co-transfected by conventional techniques with both the first and second vectors (or simply transfected by a single vector) comprising both the recombinant or synthetic light and heavy chains to create the transfected host cell of the invention. The transfected cell is then cultured by conventional techniques to produce the engineered dual targeting protein of the invention. The antigen binding protein or dual targeting protein which includes the association of both the recombinant heavy chain and/or light chain is isolated from culture and analysed by appropriate assay, such as ELISA or RIA. Similar conventional techniques may be employed to construct other dual targeting proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham (Buckinghamshire, United Kingdom) or Pharmacia (Uppsala, Sweden).

Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR) or the CMV promoter. Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the dual targeting proteins of the present invention. Host cells useful for the cloning and other manipulations of these cloning vectors are also conventional. However, cells from various strains of E. coli may be used for replication of the cloning vectors and other steps in the construction of dual targeting proteins of this invention.

Suitable host cells or cell lines for the expression of the dual targeting proteins of the invention include mammalian cells such as NS0, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs or other embodiments of the present invention (see, e.g., Plückthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and references cited therein.

The general methods by which the vectors may be constructed, the transfection methods required to produce the host cells of the invention, and culture methods necessary to produce the dual targeting protein of the invention from such host cell may all be conventional techniques. Typically, the culture method of the present invention is a serum-free culture method, usually by culturing cells serum-free in suspension. Likewise, once produced, the antigen binding proteins/dual targeting proteins of the invention may be purified from the cell culture contents according to standard procedures of the art, including ammonium sulfate precipitation, affinity columns, column chromatography, gel electrophoresis and the like. Such techniques are within the skill of the art and do not limit this invention. For example, preparation of altered antibodies are described in WO 99/58679 and WO 96/16990.

Yet another method of expression of the dual targeting proteins may utilize expression in a transgenic animal, such as described in U.S. Pat. No. 4,873,316.

This relates to an expression system using the animal's casein promoter which when transgenically incorporated into a mammal permits the female to produce the desired recombinant protein in its milk.

In a further aspect of the invention there is provided a method of producing an antigen binding proteins/dual targeting proteins of the invention which method comprises the step of culturing a host cell transformed or transfected with a vector encoding the light and/or heavy chain of the antigen binding proteins/dual targeting proteins of the invention and recovering the antigen binding proteins/dual targeting proteins thereby produced.

In accordance with the present invention there is provided a method of producing a dual targeting protein of the present invention which method comprises the steps of;
(a) providing a first vector encoding a heavy chain of the dual targeting protein,
(b) providing a second vector encoding a light chain of the dual targeting protein,
(c) transforming a mammalian host cell (e.g. CHO) with said first and second vectors;
(d) culturing the host cell of step (c) under conditions conducive to the secretion of the dual targeting protein from said host cell into said culture media;
(e) recovering the secreted dual targeting protein of step (d).

Once expressed by the desired method, the antigen binding protein/dual targeting protein is then examined for in vitro activity by use of an appropriate assay.

Presently conventional ELISA assay formats are employed to assess qualitative and quantitative binding of the antigen binding protein/dual targeting protein to its target. Additionally, other in vitro assays may also be used to verify neutralizing efficacy prior to subsequent human clinical studies performed to evaluate the persistence of the antigen binding protein/dual targeting protein in the body despite the usual clearance mechanisms.

The dose and duration of treatment relates to the relative duration of the molecules of the present invention in the human circulation, and can be adjusted by one of skill in the art depending upon the condition being treated and the general health of the patient. It is envisaged that repeated dosing (e.g. once a week or once every two weeks) over an extended time period (e.g. four to six months) maybe required to achieve maximal therapeutic efficacy.

The mode of administration of the therapeutic agent of the invention may be any suitable route which delivers the agent to the host. The dual targeting proteins, and pharmaceutical compositions of the invention are particularly useful for parenteral administration, i.e., subcutaneously (s.c.), intrathecally, intraperitoneally, intramuscularly (i.m.), intravenously (i.v.), or intranasally.

Therapeutic agents of the invention may be prepared as pharmaceutical compositions containing an effective amount of the dual targeting protein or each component of the composition of the invention as an active ingredient in a pharmaceutically acceptable carrier. In the prophylactic agent of the invention, an aqueous suspension or solution containing the composition or dual targeting protein, preferably buffered at physiological pH, in a form ready for injection is preferred. The compositions for parenteral administration will commonly comprise a solution of the dual targeting protein of the invention or a cocktail thereof dissolved in a pharmaceutically acceptable carrier, preferably an aqueous carrier. A variety of aqueous carriers may be employed, e.g., 0.9% saline, 0.3% glycine, and the like.

These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the dual targeting protein of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of a dual targeting protein of the invention. Similarly, a pharmaceutical composition of the invention for intravenous infusion could be made up to contain about 250 ml of sterile Ringer's solution, and about 1 to about 30 and preferably 5 mg to about 25 mg of a dual targeting protein of the invention per ml of Ringer's solution. Actual methods for preparing parenterally administrable compositions are well known or will be apparent to those skilled in the art and are described in more detail in, for example, Remington's Pharmaceutical Science, 15th ed., Mack Publishing Company, Easton, Pa. For the preparation of intravenously administrable dual targeting protein formulations of the invention see Lasmar U and Parkins D “The formulation of Biopharmaceutical products”, Pharma. Sci. Tech. today, page 129-137, Vol. 3 (3 Apr. 2000), Wang, W “Instability, stabilisation and formulation of liquid protein pharmaceuticals”, Int. J. Pharm 185 (1999) 129-188, Stability of Protein Pharmaceuticals Part A and B ed Ahern T. J., Manning M. C., New York, N.Y.: Plenum Press (1992), Akers, M. J. “Excipient-Drug interactions in Parenteral Formulations”, J. Pharm Sci 91 (2002) 2283-2300, Imamura, K et al “Effects of types of sugar on stabilization of Protein in the dried state”, J Pharm Sci 92 (2003) 266-274, Izutsu, Kkojima, S. “Excipient crystalinity and its protein-structure-stabilizing effect during freeze-drying”, J Pharm. Pharmacol, 54 (2002) 1033-1039, Johnson, R, “Mannitol-sucrose mixtures-versatile formulations for protein lyophilization”, J. Pharm. Sci, 91 (2002) 914-922.

Ha, E Wang W, Wang Y. j. “Peroxide formation in polysorbate 80 and protein stability”, J. Pharm Sci, 91, 2252-2264,(2002) the entire contents of which are incorporated herein by reference and to which the reader is specifically referred.

It is preferred that the therapeutic agent of the invention, when in a pharmaceutical preparation, be present in unit dose forms. The appropriate therapeutically effective dose will be determined readily by those of skill in the art. Suitable doses may be calculated for patients according to their weight, for example suitable doses may be in the range of 0.01 to 20 mg/kg, for example 0.1 to 20 mg/kg, for example 1 to 20 mg/kg, for example 10 to 20 mg/kg or for example 1 to 15 mg/kg, for example 10 to 15 mg/kg. To effectively treat conditions of use in the present invention in a human, suitable doses may be within the range of 0.01 to 1000 mg, for example 0.1 to 1000 mg, for example 0.1 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of a dual targeting protein of this invention, which may be administered parenterally, for example subcutaneously, intravenously or intramuscularly. Such dose may, if necessary, be repeated at appropriate time intervals selected as appropriate by a physician.

The dual targeting proteins described herein can be lyophilized for storage and reconstituted in a suitable carrier prior to use. This technique has been shown to be effective with conventional immunoglobulins and art-known lyophilization and reconstitution techniques can be employed.

It will be understood that the sequences described herein include sequences which are substantially identical, for example sequences which are at least 90% identical, for example which are at least 91%, or at least 92%, or at least 93%, or at least 94% or at least 95%, or at least 96%, or at least 97% or at least 98%, or at least 99% identical to the sequences described herein.

For nucleic acids, the term “substantial identity” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial identity exists when the segments will hybridize under selective hybridization conditions, to the complement of the strand.

For nucleotide and amino acid sequences, the term “identical” indicates the degree of identity between two nucleic acid or amino acid sequences when optimally aligned and compared with appropriate insertions or deletions. Alternatively, substantial identity exists when the DNA segments will hybridize under selective hybridization conditions, to the complement of the strand.

The percent identity between two sequences is a function of the number of identical positions shared by the sequences (i.e., % identity=# of identical positions/total # of positions times 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

By way of example, a polypeptide sequence of the present invention may be identical to the reference sequence encoded by SEQ ID NO: 24, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by SEQ ID NO: 24 by the numerical percent of the respective percent identity (divided by 100) and then subtracting that product from said total number of amino acids in the polypeptide sequence encoded by SEQ ID NO: 24, or:

na≦xa−(xa·y),

wherein na is the number of amino acid alterations, xa is the total number of amino acids in the polypeptide sequence encoded by SEQ ID NO: 24, and y is, for instance 0.70 for 70%, 0.80 for 80%, 0.85 for 85% etc., and wherein any non-integer product of xa and y is rounded down to the nearest integer prior to subtracting it from xa.

### Examples

### Example 1

### Construction of Dual Targeting Proteins

Design of Dual Targeting Proteins

Dual targeting proteins described herein were generated by linking a heavy chain and/or light chain of an anti-FGFR1c antibody via an optional linker to a GLP-1 agonist molecule so that the C-terminus of the agonist peptide was linked to the N-terminus of the heavy or light chain. The antibodies and antibody fusions were made by co-expression of heavy and light chains, and a list of these molecules are set out in table 1.

TABLE 1

| | Heavy Chain | Light Chain |
|---|---|---|
| Molecule Name | SEQ ID NO | SEQ ID NO |
| Ex4-FGFR1cA1H | 12 | 4 |
| Ex4-FGFR1cA1L | 2 | 14 |
| Ex4-ScrH | 20 | 8 or 24 |
| Ex4-ScrL | 6 | 22 |
| GLP-1ScrH | 26 | 8 or 24 |
| GLP-1ScrL | 6 | 28 |
| GLP1ScrH/L | 26 | 28 |
| GLP-1TVAAPSFGFR1cH | 16 | 4 |
| GLP-1TVAAPSFGFR1cL | 2 | 18 |
| Ex4-FGFR1cA1H/L | 12 | 14 |

Two versions of the light chain of the scrambled mAb were made with one amino acid difference. These two sequences are set out in SEQ ID NO:8 and SEQ ID NO:24. The amino acid difference was not believed to have any effect on the resulting antibody. The two light chains were used interchangeably, and the scrambled mAb light chains in the antibodies and antibody fusions used in the following examples may have either the light chain set out in SEQ ID NO: 8 or SEQ ID NO:24.

Molecular Biology and Expression

DNA sequences encoding the heavy and light chains of the antibodies and peptide fusions were cloned into mammalian expression vectors of the pRLN, pRLD or pTT series. The constructs made in pRLN or pRLD were transferred to pTT5 for expression in HEK293E cells.

In order to express these proteins, it is necessary to add a signal peptide sequence at the N-terminus to direct the fusion proteins for secretion. An example of a suitable signal peptide sequence is given in SEQ ID NO:33. The full length fusion protein including the signal peptide sequence can be back-translated to obtain a DNA sequence. In some cases it may be useful to codon optimise the DNA sequence for improved expression. In order to facilitate expression, a Kozak sequence and stop codons are added. In order to facilitate cloning, restriction enzyme sites can be included at the 5′ and 3′ ends. Similarly, restriction enzyme sites can also be engineered into the coding sequence to facilitate the shuffling of domains although in some cases it may be necessary to modify the amino acid sequence to accommodate a restriction site.

For mammalian expression systems, dual targeting proteins can be recovered from the supernatant, and can be purified using standard purification technologies such as Protein A sepharose.

The dual targeting proteins and combinations can then be tested in a variety of assays to assess binding to FGFR1c and GLP-1 and for biological activity in a number of assays including ELISA e.g. competition ELISA, receptor neutralisation ELISAs, BIAcore or cell-based assays which will be well known to the skilled man.

### Example 2

### FGFR1c Binding Assay

This assay was set up to test the binding of FGFR1c antibodies and dual targeting proteins of the invention to FGFR1c.

Assay plates were coated with recombinant human FGFR1c receptor (FGFR1c: Recombinant human FGFR1α (IIIc)/Fc Chimera R&D system) with 50 ul/well of receptor diluted to 1 ug/ml in coating buffer (0.2M Sodium Carbonate Buffer) and incubated overnight at 4° C. The plates were then washed 5 times with washing buffer (Phosphate Buffered Saline (PBS)+0.1% Tween20). Plates were blocked with blocking buffer (Phosphate Buffered Saline (PBS)+Bovine Serum Albumin (BSA) 1 mg/ml+0.1% Tween20) 100 μ/well and incubated at 37° C. in shaker incubator for a minimum of 30 minutes. The plates were then washed 3 times with washing buffer. Serial dilutions of test samples were made (3 fold dilutions) in blocking buffer and transferred to assay plates at 50 μl in duplicate. Plates were incubated at 37° C. in shaker incubator for 2 hours. They then were washed 5 times with washing buffer. Bound test samples were detected by polyclonal rabbit anti mouse immunoglobulin/HRP (Dako #P0260) diluted 1/1000 in blocking buffer.

50 μl/well of the detection antibody was added and incubated at 37° C. in shaker incubator for 2 hours. The plates were then washed 5 times with washing buffer. O-phenylenediamine dihydrochloride (Sigma fast OPD) was reconstituted in 20 ml H₂O, 50 μl/well was added and incubated at RT for ˜10 min. 50 μl/well of 1MH₂SO₄ was added. The plates were read at OD490 nm using the VERSAmax plate reader (Molecular Devices) and SoftmaxPro 5 software.

The following molecules were run in this assay at least twice and representative results are shown: FGFR1cA1, Ex4FGFR1A1cH, Ex4FGFR1cA1H/L, and Ex4FGFR1cA1L (FIG. 1), EX4G4S4FGFR1cH, EX4G4S4FGFR1cL, EX4ASTKFGFR1cH, EX4ASTKFGFR1cL, Ex4 FGFR1cA1H, and FGFR1cA1 (FIG. 2), EX4TVAAPSFGFR1cL, GLP1TVAAPSFGFR1cL, EX4TVAAPSFGFR1cH, GLP1TVAAPSFGFR1cH, G4S2FGFR1cL, G4S2FGFR1cH. (FIG. 3). Additionally, an FGFR1b antibody which was known not to bind to FGFR1c was run as a negative control (FIG. 1).

### Example 3

### FGFR1c Receptor Binding Inhibition Assay

This assay was set up to test the inhibition of ligand binding (FGF) to its receptor (FGFR1c) in the presence of FGFR1c antibodies and dual targeting proteins of the invention.

Assay plates were coated with recombinant human basic fibroblast growth factor (FGF-basic 157aa) (R&D Systems #234-FSE/CF) at 4 μg/ml in coating buffer (0.2M Sodium Carbonate Buffer). 50 μl/well of this mixture was incubated overnight at 4° C. The plates were then washed 5 times with washing buffer (Phosphate Buffered Saline (PBS)+0.1% Tween20). Heparan sulphate proteoglycan (HSPG) in blocking buffer (Phosphate Buffered Saline (PBS)+Bovine Serum Albumin (BSA) 1 mg/ml+0.1% Tween20) at 1 ug/ml was added in 100 μl/well and incubated at 37° C. in shaker incubator for a minimum of 30 minutes (HSPG binding protects FGF from denaturation and proteolytic degradation).

The plates were then washed 3 times with washing buffer. Serial dilutions of standards and samples were made in blocking buffer.

30 ug/ml of receptor (Recombinant human FGFR1α (IIIc)/Fc Chimera) was made in blocking buffer. Reaction mixes were made by making 150 μl (5 ul receptor/145 ul mAbs) of each dilution of mAbs. 50 μl/well of each reaction mix was added to appropriate wells in duplicate and incubated at 37° C. in shaker incubator for 2 hours. The plates then were washed 5 times with washing buffer. Anti-Human Polyvalent Immunoglobulins—Peroxidase antibody was diluted in blocking buffer 1:1000, 50 ul/well of this mixture was incubated at 37° C. in shaker incubator for 2 hours. The plates were then washed 5 times with washing buffer. O-phenylenediamine dihydrochloride (Sigma fast OPD) was reconstituted in 20 ml H₂O, 50 μl/well was added and incubated at RT for ˜10min. 50 μl/well of 1M H₂SO₄ was added. The plates were read at OD490 nm using the VERSAmax plate reader (Molecular Devices) and SoftmaxPro 5 software.

The following molecules were run in this assay: FGFR1cA1, Ex4FGFR1cA1H, Ex4FGFR1cA1L and Ex4FGFR1cA1H/L. Additionally, an FGFR1b antibody which was known not to bind to FGFR1c was run as a negative control. The results are shown in FIG. 4.

### Example 4

### GLP-1 Binding Assay

CHO 6CRE GLP1 R cells were rapidly defrosted by half immersing the vial(s) in a 37° C. water bath, and the contents of the vial(s) transferred to a 50 ml falcon tube and 10 ml RPMI (phenol red free) assay media (Sigma, cat# R7509)+2 mM L-glutamine (Gibco, cat # 25030)+15 mM HEPES (Sigma, cat # H0887) added per vial. After counting and centrifugation at 1200 rpm for 5 minutes cells were resuspended in the appropriate volume of RPMI assay media to give 1×10⁶ cells per ml and 50 μl dispensed into each well of a white 96 well flat bottom tissue culture plate (Costar 96 well tissue culture plate, white sterile, cat # 3917). Cells were incubated overnight at 37° C./5%CO2. Next day cells were removed from incubator and 50 μl of previously prepared control/sample was added to wells and plate was returned to incubator for 3 hours 37° C. and 5% CO2.

After the incubation time 50 μl of Bright-Glo Luciferase reagent was added to all wells and the plate was incubated at room temperature for 3 mins to allow cell lysis to occur. The luminescence (counts per second) was read using the M5e microplate reader, reading each well for 0.1 sec. CPS of the background wells containing cells only, was subtracted from all other wells. The control wells (GLP-1(7-36) or Exendin-4) should exhibit maximum stimulation at the highest concentrations. Concentration effect curves of the unknown samples are fitted from which the EC50 is calculated with use of Graph Pad Prism or ExcelFit software.

Results of the molecules tested in this assay are shown in table 2.

TABLE 2

| | Average EC50 |
|---|---|
| Antibody Fusion molecules | (pM) |
| Ex4LScr | 116.7 (n = 5) |
| Ex4HScr | 152.9 (n = 7) |
| GLP1LScr | 889.4 (n = 3) |
| GLP1HScr | 484.8 (n = 3) |
| Ex4-FGFR1cL | 108.7 (n = 1) |
| Ex4-FGFR1cH | 117.0 (n = 2) |
| Ex4-FGFR1cHL | 455.4 (n = 1) |
| GLP1ScrHL | 475.3 (n = 1) |

### Example 5

### Biacore Assay

Anti-human IgG (Biacore BR-1008-39) was immobilised on a CM5 chip by primary amine coupling. The anti human IgG surface was used to capture Fc tagged FGFR1c receptor. After the receptor capture, antibody was passed over at 256, 64, 16, 4, 1 and 0.25 nM with a 0 nM (i.e. buffer alone) injection used to double reference the binding data, double referencing helps remove machine artefacts and corrects for any baseline drift.

After each antibody concentration binding sensorgram had been generated, the captured receptor was removed from the anti-human IgG surface by using 3M MgCl₂, the receptor was then captured again for the next concentration of antibody to be passed over. The run was carried out using HBS-EP and run at 25° c. The work was carried out on the Biacore T100 machine and data was fitted to the 1:1 and Bivalent models inherent to the machines analysis software. Table 4 details the kinetic parameters obtained for the Bivalent model whilst Table 5 shows the data obtained from the 1:1 model.

TABLE 4
Bivalent Model Data

| Construct | ka1 | kd1 | KD1 (nM) |
|---|---|---|---|
| FGFR1c(A1) | 2.998E+5 | 8.864E−4 | 2.96 |
| Ex4FGFR1cH | 4.596E+4 | 1.228E−3 | 26.7 |
| Ex4FGFR1cL | 9.648E+4 | 2.354E−2 | 244 |
| Ex4FGFR1cHL | 5.530E+3 | 5.529E−3 | 999.8 |

Data only describes the first interaction of the Bivalent binding event.

TABLE 5
1:1 Model Data

| Construct | ka | kd | KD (nM) |
|---|---|---|---|
| FGFR1c(A1) | 3.977E+5 | 6.294E−4 | 1.58 |
| Ex4FGFR1cH | 8.347E+4 | 1.008E−3 | 12.1 |
| Ex4FGFR1cL | 1.111E+5 | 1.479E−3 | 13.3 |
| Ex4FGFR1cHL | 1.141E+4 | 2.256E−3 | 198 |

### Example 6

### Mouse Diet Induced Obesity (DIO) Model

Obesity was induced in 6-8 week old singly housed male C57bl6/J mice by feeding with a defined diet delivering 45% kcal from Fat and 20% kcal protein (Land of Lakes Purina Feed LLC, St Louis, Mo.) for 18-25 weeks. A second group of control mice from the same batch was fed for the same period with a matched 10% kcal fat/20% kcal protein diet. Standardised environmental enrichment was provided. Mice were selected for dosing based on an attained mean body weight of 47-50 g per dose group of eight mice. Mice were weighed twice weekly and diet consumption monitored daily throughout the study. In addition, proportions of fat and lean tissue were measured prior to and during study by quantitive magnetic resonance (qMR) using an EchoMRI-700™ scanner (Echo MRI, Houston, Tex.). Each mouse was placed in a holding tube, inserted into the scanning chamber and a minimum of three 52 second scans performed. Following initial weight, diet consumption and qMR measurements mice were dosed intraperitoneally (IP) at 0.1 ml/10 g body weight with 10 mg/Kg of either of the following molecules: Scrambled mAb (SEQ ID NO: 6 and SEQ ID NO: 8 or SEQ ID NO:24), Ex4ScrH (SEQ ID NO:20 and SEQ ID NO: 8 or SEQ ID NO:24), FGFR1cA1 (SEQ ID NO:2 and SEQ ID NO:4), Ex4FGFR1cA1H (SEQ ID NO: 12 and SEQ ID NO: 4) or a combination of FGFR1cA1 (SEQ ID NO:2 and SEQ ID NO:4) and Ex4ScrH (SEQ ID NO:20 and SEQ ID NO: 8 or SEQ ID NO:24). Further groups were dosed IP with Exendin-4 (Ex-4) peptide (SEQ ID NO:) (E7144, Sigma, Gillingham, Dorset, UK)) or Phosphate Buffered saline (pH 7.2) according to the following schedule:

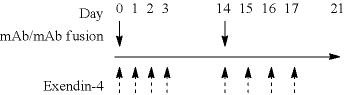

The results are set out in FIGS. 5, 6 and 7.

### a) Diet Consumption (FIG. 5)

Maximum effects on diet consumption were observed within three days following each dose, with the greatest reduction compared with the Scr mAb achieved with both the mixture of FGFR1cA1/Ex4ScrH mAbs (day 1-4 feeding reduced by 2.0 g/day, p<0.0001; days 14-21 reduced by 1.6 g/day, p<0001) and the Ex4FGFR1cA1H fusion mAb (days 0-3 feeding reduced by 1.9 g/day, p<0.0001; days 14-21 reduced by 1.4 g/day, p<0001. Reduction in feeding with both the mixture and the fusion was significantly greater than for FGFR1cA1 mAb alone (days 0-3 p<0.0001, days 14-21 p=0.0003 and days 0-3 p<0.0003, days 14-21 p=0.0088 respectively). In addition an analysis of the day 14-21 feeding data following the second dose of FGFR1cA1/Ex4ScrH mAb mixture showed an unexpected synergistic effect vs FGFR1c alone (p=0.0317) showing a reduction in feeding by a further 0.38 g/day over the additive effect of both antibodies alone.

Reduction in feeding in mice treated with Ex4Scr mAb was more transient (day 1-4 feeding reduced by 0.87 g/day, p<0.0001; day 14-21 reduced by 0.17 g/day, p=0.4979). Diet consumption recovered more rapidly in mice dosed with the Ex4FGFR1cA1H fusion mAb than in the case of the FGFR1cA1/Ex4ScrH mAb mixture, possibly reflecting the 10 fold reduction in affinity to FGFR1c receptors of the fusion observed in vitro and following the pattern of recovery following dosing with the Ex4Scr mAb (Day 0-6 vs. FGFR1cA1 mab p=0.0179).

### b) Change in Body Weight (FIG. 6)

Cumulative weight reduction on day 3 following the first dose of both the mixture of FGFR1cA1/Ex4ScrH mAbs and the Ex4FGFR1cA1H fusion mAb vs the Scr mAb were both highly significant (p<0.0001, c. 6.4 g) and also vs. the FGFR1cA1 mAb (p<0.0001, 1.89 and 1.79 g respectively). Following the second dose both the mixture and the fusion showed a similar reduction in weight vs the Scr mAb (Days 14-21, p<0.0001, 19 and 12 g respectively). The antibody mixture produced a significant increase in weight loss compared with the FGFR1cA1 mAb following the second dose (Days 14-21, p<0.0001, 3.6 g).

### c) Body Fat/Lean Tissue (FIG. 7)

Loss of fat tissue following initial doses of FGFR1cA1/Ex4ScrH mAbs, the Ex4FGFR1cA1 H fusion or FGFR1cA1 alone compared with the Scr mAb were similar (p <0.0001, 29.0%,24.9% and 26.5% respectively), however three days following the second dose of FGFR1cA1/Ex4ScrH mAbs mixture a fat tissue loss of 65.7% was achieved which was 10% greater than the loss achieved with FGFR1cA1 mAb alone (p<0.0001).

Some lean tissue loss also occurred in groups dosed with FGFR1c mAb based combinations, however loss in the FGFR1cA1/Ex4ScrH mAbs mixture dosed group on completion of the experiment (day 21) was 17% (5.1% more than FGFR1c mAb alone p=0.0195) compared with a fat tissue loss of 71% vs Scr mAb on day 21.

### Example 7

### Mouse Diet Induced Obesity (DIO) Model Dose Range Study

The DIO model as described in example 6 was used except that mice were weighed daily. Following initial weight, diet consumption and qMR measurements mice were dosed IP at 0.1 ml/10 g body weight with 10, 3 or 1 mg/Kg of either of the following molecules: Ex4ScrH (SEQ ID NO:20 and SEQ ID NO: 8 or SEQ ID NO:24), FGFR1cA1 (SEQ ID NO:2 and SEQ ID NO:4), Ex4FGFR1cA1H (SEQ ID NO: 12 and SEQ ID NO: 4) or a combination of FGFR1cA1 (SEQ ID NO:2 and SEQ ID NO:4) and Ex4ScrH (SEQ ID NO:20 and SEQ ID NO: 8 or SEQ ID NO:24). Further groups were dosed IP with Scrambled mAb (SEQ ID NO: 6 and SEQ ID NO: 8 or SEQ ID NO:24) (10 mg/Kg), Exendin-4 (Ex-4) peptide (SEQ ID NO:) (RP10874,GenScript, Piscataway, N.J., USA) (1 mg/Kg: an approximately 25:1 molar ration difference compared to Ex4ScrH at 10 mg/Kg) or Phosphate Buffered saline (pH 7.2) according to the following schedule:

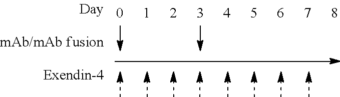

The results are set out in FIGS. 9,10 and 11.

### a) Diet Consumption (FIG. 9)

Maximum effects on diet consumption were observed within three days following the first dose, with the greatest reduction compared with the Scr mAb achieved with both the mixture of FGFR1cA1/Ex4ScrH mAbs (day 2 feeding reduced by 3.1 g/day in groups dosed with 10 or 3 mg/Kg and by 2.5 g/day in the 1 mg/Kg dosed group) and the Ex4FGFR1cA1 H fusion mAb (day 2 feeding reduced by 3, 2.67 or 1.89 g/day in groups dosed with 10, 3 or 1 mg/Kg respectively). Reduction in food consumption with both the mixture and the fusion was greater than with FGFR1cA1 mAb alone (where day 2 feeding was reduced by 2.3, 1.86 or 1.61 g/day in groups dosed with 10, 3 or 1 mg/Kg respectively). The greatest reduction in feeding in the Ex4ScrH mAb dosed group was achieved on day 1 following the first dose (feeding reduced by 2.26, 2.1 or 2.12 g/day in groups dosed with 10, 3 or 1 mg/Kg respectively) but the effect was more transient than with the other groups and had already begun to increase by day 2, prior to the second dose (feeding reduced on day 2 by 1.96, 1.67 or 1.79 g/day in groups dosed with 10, 3 or 1 mg/Kg respectively). Following the second dose overall reduction in feeding was sustained at levels prior to the second dose, with the Ex4ScrH mAb dosed group showing a reduction in feeding on day 4 compared to levels at day 3 (prior to the second dose), demonstrating that more frequent dosing is able to overcome the more transient nature of the effect of Ex4ScrH mAb in vivo.

### b) Change in Body Weight (FIG. 10)

Weight reduction on day 5 following the second dose of both the mixture of FGFR1cA1/Ex4ScrH mAbs and the Ex4FGFR1cA1 H fusion mAb vs the Scr mAb were both high (c. 13.5 g and 14.5 g respectively for the 10 mg/Kg groups) whereas weight reduction in the group dosed with 10 mg/Kg FGFR1cA1 mAb was the equivalent to weight reductions achieved with a 3 fold lower dose of either the mixture or the fusion (11, 11.49 and 10.66 g respectively).

### c) Body Fat/Lean Tissue (FIG. 11)

Four days following the second dose of 10 mg/KgKg FGFR1cA1/Ex4ScrH mAbs mixture or the Ex4FGFR1cA1 H fusion mAb a fat tissue loss of c. 38% was achieved which was c. 15% greater than the loss achieved with 10 mg/Kg FGFR1cA1 mAb alone (32.27%) and similar differences were observed with lower doses.

Some lean tissue loss also occurred in groups dosed with FGFR1c mAb based combinations and in groups dosed with the FGFR1c and Ex4ScrH mabs dosed alone, with a maximum of 15.66% with the group given 10 mg/Kg Ex4FGFR1cA1H fusion mAb, however the lean tissue losses were reduced at lower dose levels.

### SEQUENCES

TABLE 3

| | Sequence Identifier | |
|---|---|---|
| | (SEQ ID NO) | |
| Description | Polynucleotide | Amino acid |
| Chimeric FGFR1c antibody heavy chain | 1 | 2 |
| Chimeric FGFR1c antibody light chain | 3 | 4 |
| Scr (scrambled) antibody heavy chain | 5 | 6 |
| Scr (scrambled) antibody light chain | 7 | 8 |
| Exendin 4 | — | 9 |
| GLP-1 | — | 10 |
| Exendin 4-G4S2-FGFR1c antibody heavy chain | 11 | 12 |
| Exendin 4- G4S2-FGFR1c antibody light chain | 13 | 14 |
| GLP-1- TVAAPS-FGFR1c antibody heavy chain | 15 | 16 |
| GLP-1-TVAAPS-FGFR1c antibody light chain | 17 | 18 |
| Exendin 4-G4S2-Scr (scrambled) antibody heavy | 19 | 20 |
| chain | | |
| Exendin 4- G4S2-Scr (scrambled) antibody light | 21 | 22 |
| chain | | |
| Scr (scrambled) antibody Alternative light chain | 23 | 24 |
| GLP-1- G4S2-Scr (scrambled) antibody heavy chain | 25 | 26 |
| GLP-1- G4S2-Scr (scrambled) antibody light chain | 27 | 28 |
| FGFR1c antibody VH | 29 | 30 |
| FGFR1c antibody VL | 31 | 32 |
| Mammalian signal sequence | — | 33 |
| Linker G4S4 | — | 34 |
| Linker TVAAPS | — | 35 |
| Linker ASTK | — | 36 |
| Linker G4S | — | 37 |

SEQ ID NO: 1
(FGFR1c antibody heavy chain)
CAGGTGCAGCTGGTGCAGAGCGGCGCTGAGGTGAAGAAGCCCGGCTCCTCCGTCAAGGTCAGCTGCAAG
GCGAGCGGGCAGACATTCACCGGATACTACATGCATTGGGTGCGCCAGGCCCCGGGGCAGGGGCTCGAG
TGGATGGGGAGAATCATCCCCATCCTGGGCATCGCTCAGAAGTTCCAGGGACGCGTGACCATCACCGCC
GACAAATCCACCAGCACCGCCTACATGGAACTGAGCTCCCTGCGCTCCGAGGACACCGCCGTGTATTAT
TGCGCCCGCGGGGGCGACCTGGGCGGCATGGACGTGTGGGGCCAGGGCACACTAGTCACCGTCTCCTCA
GCCAAAACAACAGCCCCATCGGTCTATCCACTGGCCCCTGTGTGTGGAGATACAACTGGCTCCTCGGTG
ACTCTAGGATGCCTGGTCAAGGGTTATTTCCCTGAGCCAGTGACCTTGACCTGGAACTCTGGATCCCTG
TCCAGTGGTGTGCACACCTTCCCAGCTGTCCTGCAGTCTGACCTCTACACCCTCAGCAGCTCAGTGACT
GTAACCTCGAGCACCTGGCCCAGCCAGTCCATCACCTGCAATGTGGCCCACCCGGCAAGCAGCACCAAG
GTGGACAAGAAAATTGAGCCCAGAGGGCCCACAATCAAGCCCTGTCCTCCATGCAAATGCCCAGCACCT
AACCTCGCGGGTGCACCATCCGTCTTCATCTTCCCTCCAAAGATCAAGGATGTACTCATGATCTCCCTG
AGCCCCATAGTCACATGTGTGGTGGTGGATGTGAGCGAGGATGACCCAGATGTCCAGATCAGCTGGTTT
GTGAACAACGTGGAAGTACACACAGCTCAGACACAAACCCATAGAGAGGATTACAACAGTACTCTCCGG
GTGGTCAGTGCCCTCCCCATCCAGCACCAGGACTGGATGAGTGGCAAGGAGTTCAAATGCAAGGTCAAC
AACAAAGACCTCCCAGCGCCCATCGAGAGAACCATCTCAAAACCCAAAGGGTCAGTAAGAGCTCCACAG
GTATATGTCTTGCCTCCACCAGAAGAAGAGATGACTAAGAAACAGGTCACTCTGACCTGCATGGTCACA
GACTTCATGCCTGAAGACATTTACGTGGAGTGGACCAACAACGGGAAAACAGAGCTAAACTACAAGAAC
ACTGAACCAGTCCTGGACTCTGATGGTTCTTACTTCATGTACAGCAAGCTGAGAGTGGAAAAGAAGAAC
TGGGTGGAAAGAAATAGCTACTCCTGTTCAGTGGTCCACGAGGGTCTGCACAATCACCACACGACTAAG
AGCTTCTCCCGGACTCCGGGTAAA
SEQ ID NO: 2
(FGFR1c antibody heavy chain)
QVQLVQSGAEVKKPGSSVKVSCKASGQTFTGYYMHWVRQAPGQGLEWMGRIIPILGIAQKFQGRVTITA
DKSTSTAYMELSSLRSEDTAVYYCARGGDLGGMDVWGQGTLVTVSSAKTTAPSVYPLAPVCGDTTGSSV
TLGCLVKGYFPEPVTLTWNSGSLSSGVHTFPAVLQSDLYTLSSSVTVTSSTWPSQSITCNVAHPASSTK
VDKKIEPRGPTIKPCPPCKCPAPNLAGAPSVFIFPPKIKDVLMISLSPIVTCVVVDVSEDDPDVQISWF
VNNVEVHTAQTQTHREDYNSTLRVVSALPIQHQDWMSGKEFKCKVNNKDLPAPIERTISKPKGSVRAPQ
VYVLPPPEEEMTKKQVTLTCMVTDFMPEDIYVEWTNNGKTELNYKNTEPVLDSDGSYFMYSKLRVEKKN
WVERNSYSCSVVHEGLHNHHTTKSFSRTPGK
SEQ ID NO: 3
(FGFR1c antibody light chain)
GAGATCGTGCTGACCCAGAGCCCCCTCTCGCTGCCCGTGACCCCCGGGGAGCCCGCCAGCATCTCCTGC
CGCAGTAGCCAGAGCCTGAGGCATTCCAATGGCTACAACTACCTGGACTGGTACCTGCAGAAACCCGGC
CAGAGCCCCCAGCTGCTCATCTACCTGGCGAGTAACCGCGCCAGCGGGGTGCCCGACCGCTTCAGCGGC
TCCGGCAGTGGAACCGACTTCACCCTGAAGATCTCCCGCGTGGAGGCGGAGGACGTGGGGGTGTATTAC
TGTATGCAGGCCCTCCAGATCCCCCCCACGTTCGGCCCCGGCACCAAGGTGGACATCAAACGCACCGTC
GCCGCCCCGACCGTGAGCATTTTCCCTCCCAGCTCCGAGCAGCTGACGTCCGGCGGCGCCTCTGTGGTG
TGCTTCCTCAACAACTTCTACCCCAAGGACATCAACGTGAAGTGGAAGATCGACGGCTCCGAGAGACAG
AACGGCGTGCTGAACAGCTGGACCGACCAGGACAGCAAGGACTCCACCTACAGTATGAGCTCCACCCTG
ACCCTGACCAAGGACGAGTACGAGAGGCATAACTCTTATACCTGCGAGGCGACCCATAAGACCAGCACC
TCCCCCATCGTCAAGAGCTTCAACCGCAACGAATGC
SEQ ID NO: 4
(FGFR1c antibody light chain)
EIVLTQSPLSLPVTPGEPASISCRSSQSLRHSNGYNYLDWYLQKPGQSPQLLIYLASNRASGVPDRFSG
SGSGTDFTLKISRVEAEDVGVYYCMQALQIPPTFGPGTKVDIKRTVAAPTVSIEPPSSEQLTSGGASVV
CFLNNFYPKDINVKWKIDGSERQNGVLNSWTDQDSKDSTYSMSSTLTLTKDEYERHNSYTCEATHKTST
SPIVKSFNRNEC
SEQ ID NO: 5
(Scrambled antibody heavy chain)
CAGGTCCAATTAGTGCAATCTGGGTCTGAGTTGAAGAAGCCTGGGGCCTCAGTGAAGGTTTCCTGCAAG
GCCTCTGGATACACCTTCACTAACTATGGAATGAACTGGGTGCGACAGGCCCCTGGACAAGGGCTCGAG
TGGATGGGATGGATAAACACCAGAAATGGAAAGTCAACATATGTTGATGACTTCAAGGGGCGGTTTGTC
TTCTCCTTGGACACCTCTGTCAGCACGGCATATCTACAGATCAGCAGCCTAAAGGCTGACGACACTGCA
GTGTATTACTGTGCGAGAGAAGGGAATATGGATGGTTACTTCCCTTTTACTTACTGGGGCCAGGGTACA
CTAGTCACCGTCTCCTCAGCCAAAACAACAGCCCCATCGGTCTATCCACTGGCCCCTGTGTGTGGAGAT
ACAACTGGCTCCTCGGTGACTCTAGGATGCCTGGTCAAGGGTTATTTCCCTGAGCCAGTGACCTTGACC
TGGAACTCTGGATCCCTGTCCAGTGGTGTGCACACCTTCCCAGCTGTCCTGCAGTCTGACCTCTACACC
CTCAGCAGCTCAGTGACTGTAACCTCGAGCACCTGGCCCAGCCAGTCCATCACCTGCAATGTGGCCCAC
CCGGCAAGCAGCACCAAGGTGGACAAGAAAATTGAGCCCAGAGGGCCCACAATCAAGCCCTGTCCTCCA
TGCAAATGCCCAGCACCTAACCTCGCGGGTGCACCATCCGTCTTCATCTTCCCTCCAAAGATCAAGGAT
GTACTCATGATCTCCCTGAGCCCCATAGTCACATGTGTGGTGGTGGATGTGAGCGAGGATGACCCAGAT
GTCCAGATCAGCTGGTTTGTGAACAACGTGGAAGTACACACAGCTCAGACACAAACCCATAGAGAGGAT
TACAACAGTACTCTCCGGGTGGTCAGTGCCCTCCCCATCCAGCACCAGGACTGGATGAGTGGCAAGGAG
TTCAAATGCAAGGTCAACAACAAAGACCTCCCAGCGCCCATCGAGAGAACCATCTCAAAACCCAAAGGG
TCAGTAAGAGCTCCACAGGTATATGTCTTGCCTCCACCAGAAGAAGAGATGACTAAGAAACAGGTCACT
CTGACCTGCATGGTCACAGACTTCATGCCTGAAGACATTTACGTGGAGTGGACCAACAACGGGAAAACA
GAGCTAAACTACAAGAACACTGAACCAGTCCTGGACTCTGATGGTTCTTACTTCATGTACAGCAAGCTG
AGAGTGGAAAAGAAGAACTGGGTGGAAAGAAATAGCTACTCCTGTTCAGTGGTCCACGAGGGTCTGCAC
AATCACCACACGACTAAGAGCTTCTCCCGGACTCCGGGTAAA
SEQ ID NO: 6
(Scrambled antibody heavy chain)
QVQLVQSGSELKKPGASVKVSCKASGYTFTNYGMNWVRQAPGQGLEWMGWINTRNGKSTYVDDFKGRFV
FSLDTSVSTAYLQISSLKADDTAVYYCAREGNMDGYFPFTYWGQGTLVTVSSAKTTAPSVYPLAPVCGD
TTGSSVTLGCLVKGYFPEPVTLTWNSGSLSSGVHTFPAVLQSDLYTLSSSVTVTSSTWPSQSITCNVAH
PASSTKVDKKIEPRGPTIKPCPPCKCPAPNLAGAPSVFIFPPKIKDVLMISLSPIVTCVVVDVSEDDPD
VQISWFVNNVEVHTAQTQTHREDYNSTLRVVSALPIQHQDWMSGKEFKCKVNNKDLPAPIERTISKPKG
SVRAPQVYVLPPPEEEMTKKQVTLTCMVTDFMPEDIYVEWTNNGKTELNYKNTEPVLDSDGSYFMYSKL
RVEKKNWVERNSYSCSVVHEGLHNHHTTKSFSRTPGK
SEQ ID NO: 7
(Scrambled antibody light chain)
GATATTGTCATGACTCAGTCTCCATCATCCCTGTCCGCATCAGTAGGAGACAGGGTCACCATCACCTGC
AAGGCTTCTCAGAATGTGGGTACTAATGTAGCCTGGTATCAACAGAAACCAGGGAAAGCTCCTAAAGCA
CTGATTTACTCGGCATCCTATCGGTACAGTGGAGTCCCTGATCGCTTCTCAGGCAGTGGATCCGGGACA
GATTTCACTCTCACCATCAGCAGTCTGCAGCCTGAAGACTTCGCAACGTATTACTGTCAGCAATATAAC
AGCTATCCTCTCACGTTCGGTGGTGGTACCAAGGTGGAAATAAAACGTACGGATGCTGCACCGACTGTA
TCCATCTTCCCACCATCCAGTGAGCAGTTAACATCTGGAGGTGCCTCAGTCGTGTGCTTCTTGAACAAC
TTCTACCCCAAAGACATCAATGTCAAGTGGAAGATTGATGGCAGTGAACGACAAAATGGCGTCCTGAAC
AGTTGGACTGATCAGGACAGCAAAGACAGCACCTACAGCATGAGCAGCACCCTCACGTTGACCAAGGAC
GAGTATGAACGACATAACAGCTATACCTGTGAGGCCACTCACAAGACATCAACTTCACCCATTGTCAAG
AGCTTCAACAGGAATGAGTGT
SEQ ID NO: 8
(Scrambled antibody light chain)
DIVMTQSPSSLSASVGDRVTITCKASQNVGTNVAWYQQKPGKAPKALIYSASYRYSGVPDRFSGSGSGT
DFTLTISSLQPEDFATYYCQQYNSYPLTFGGGTKVEIKRTDAAPTVSIFPPSSEQLTSGGASVVCFLNN
FYPKDINVKWKIDGSERQNGVLNSWTDQDSKDSTYSMSSTLTLTKDEYERHNSYTCEATHKTSTSPIVK
SFNRNEC
SEQ ID NO: 9
(Exendin 4)
HGEGTFTSDLSKQMEEEAVRLFIEWLKNGGPSSGAPPPSG
SEQ ID NO: 10
(GLP-1)
HGEGTFTSDVSSYLEGQAAKEFIAWLVKGR
SEQ ID NO: 11
(Exendin 4-G4S2-FGFR1c antibody heavy chain)
CATGGGGAGGGCACTTTCACTAGCGACCTGAGCAAGCAGATGGAAGAAGAGGCCGTGAGGCTGTTCATT
GAGTGGCTCAAGAACGGAGGCCCCTCCTCCGGCGCCCCCCCCCCTAGCGGCGGATCCGGAGGCGGGGGC
AGTGGCGGGGGAGGTAGCGGTCAGGTGCAGCTGGTGCAGAGCGGCGCTGAGGTGAAGAAGCCCGGCTCC
TCCGTCAAGGTCAGCTGCAAGGCGAGCGGGCAGACATTCACCGGATACTACATGCATTGGGTGCGCCAG
GCCCCGGGGCAGGGGCTCGAGTGGATGGGGAGAATCATCCCCATCCTGGGCATCGCTCAGAAGTTCCAG
GGACGCGTGACCATCACCGCCGACAAATCCACCAGCACCGCCTACATGGAACTGAGCTCCCTGCGCTCC
GAGGACACCGCCGTGTATTATTGCGCCCGCGGGGGCGACCTGGGCGGCATGGACGTGTGGGGCCAGGGC
ACACTAGTCACCGTCTCCTCAGCCAAAACAACAGCCCCATCGGTCTATCCACTGGCCCCTGTGTGTGGA
GATACAACTGGCTCCTCGGTGACTCTAGGATGCCTGGTCAAGGGTTATTTCCCTGAGCCAGTGACCTTG
ACCTGGAACTCTGGATCCCTGTCCAGTGGTGTGCACACCTTCCCAGCTGTCCTGCAGTCTGACCTCTAC
ACCCTCAGCAGCTCAGTGACTGTAACCTCGAGCACCTGGCCCAGCCAGTCCATCACCTGCAATGTGGCC
CACCCGGCAAGCAGCACCAAGGTGGACAAGAAAATTGAGCCCAGAGGGCCCACAATCAAGCCCTGTCCT
CCATGCAAATGCCCAGCACCTAACCTCGCGGGTGCACCATCCGTCTTCATCTTCCCTCCAAAGATCAAG
GATGTACTCATGATCTCCCTGAGCCCCATAGTCACATGTGTGGTGGTGGATGTGAGCGAGGATGACCCA
GATGTCCAGATCAGCTGGTTTGTGAACAACGTGGAAGTACACACAGCTCAGACACAAACCCATAGAGAG
GATTACAACAGTACTCTCCGGGTGGTCAGTGCCCTCCCCATCCAGCACCAGGACTGGATGAGTGGCAAG
GAGTTCAAATGCAAGGTCAACAACAAAGACCTCCCAGCGCCCATCGAGAGAACCATCTCAAAACCCAAA
GGGTCAGTAAGAGCTCCACAGGTATATGTCTTGCCTCCACCAGAAGAAGAGATGACTAAGAAACAGGTC
ACTCTGACCTGCATGGTCACAGACTTCATGCCTGAAGACATTTACGTGGAGTGGACCAACAACGGGAAA
ACAGAGCTAAACTACAAGAACACTGAACCAGTCCTGGACTCTGATGGTTCTTACTTCATGTACAGCAAG
CTGAGAGTGGAAAAGAAGAACTGGGTGGAAAGAAATAGCTACTCCTGTTCAGTGGTCCACGAGGGTCTG
CACAATCACCACACGACTAAGAGCTTCTCCCGGACTCCGGGTAAA
SEQ ID NO: 12
(Exendin 4-G4S2-FGFR1c antibody heavy chain)
HGEGTFTSDLSKQMEEEAVRLFIEWLKNGGPSSGAPPPSGGSGGGGSGGGGSGQVQLVQSGAEVKKPGS
SVKVSCKASGQTFTGYYMHWVRQAPGQGLEWMGRIIPILGIAQKFQGRVTITADKSTSTAYMELSSLRS
EDTAVYYCARGGDLGGMDVWGQGTLVTVSSAKTTAPSVYPLAPVCGDTTGSSVTLGCLVKGYFPEPVTL
TWNSGSLSSGVHTFPAVLQSDLYTLSSSVTVTSSTWPSQSITCNVAHPASSTKVDKKIEPRGPTIKPCP
PCKCPAPNLAGAPSVFIFPPKIKDVLMISLSPIVTCVVVDVSEDDPDVQISWFVNNVEVHTAQTQTHRE
DYNSTLRVVSALPIQHQDWMSGKEFKCKVNNKDLPAPIERTISKPKGSVRAPQVYVLPPPEEEMTKKQV
TLTCMVTDFMPEDIYVEWTNNGKTELNYKNTEPVLDSDGSYFMYSKLRVEKKNWVERNSYSCSVVHEGL
HNHHTTKSFSRTPGK
SEQ ID NO: 13
(Exendin 4-G4S2-FGFR1c antibody light chain)
CATGGGGAGGGCACTTTCACTAGCGACCTGAGCAAGCAGATGGAAGAAGAGGCCGTGAGGCTGTTCATT
GAGTGGCTCAAGAACGGAGGCCCCTCCTCCGGCGCCCCCCCCCCTAGCGGCGGATCCGGAGGCGGGGGC
AGTGGCGGGGGAGGTAGCGGTGAGATCGTGCTGACCCAGAGCCCCCTCTCGCTGCCCGTGACCCCCGGG
GAGCCCGCCAGCATCTCCTGCCGCAGTAGCCAGAGCCTGAGGCATTCCAATGGCTACAACTACCTGGAC
TGGTACCTGCAGAAACCCGGCCAGAGCCCCCAGCTGCTCATCTACCTGGCGAGTAACCGCGCCAGCGGG
GTGCCCGACCGCTTCAGCGGCTCCGGCAGTGGAACCGACTTCACCCTGAAGATCTCCCGCGTGGAGGCG
GAGGACGTGGGGGTGTATTACTGTATGCAGGCCCTCCAGATCCCCCCCACGTTCGGCCCCGGCACCAAG
GTGGACATCAAACGCACCGTCGCCGCCCCGACCGTGAGCATTTTCCCTCCCAGCTCCGAGCAGCTGACG
TCCGGCGGCGCCTCTGTGGTGTGCTTCCTCAACAACTTCTACCCCAAGGACATCAACGTGAAGTGGAAG
ATCGACGGCTCCGAGAGACAGAACGGCGTGCTGAACAGCTGGACCGACCAGGACAGCAAGGACTCCACC
TACAGTATGAGCTCCACCCTGACCCTGACCAAGGACGAGTACGAGAGGCATAACTCTTATACCTGCGAG
GCGACCCATAAGACCAGCACCTCCCCCATCGTCAAGAGCTTCAACCGCAACGAATGC
SEQ ID NO: 14
(Exendin 4-G4S2-FGFR1c antibody light chain)
HGEGTFTSDLSKQMEEEAVRLFIEWLKNGGPSSGAPPPSGGSGGGGSGGGGSGEIVLTQSPLSLPVTPG
EPASISCRSSQSLRHSNGYNYLDWYLQKPGQSPQLLIYLASNRASGVPDRFSGSGSGTDFTLKISRVEA
EDVGVYYCMQALQIPPTFGPGTKVDIKRTVAAPTVSIFPPSSEQLTSGGASVVCFLNNFYPKDINVKWK
IDGSERQNGVLNSWTDQDSKDSTYSMSSTLTLTKDEYERHNSYTCEATHKTSTSPIVKSFNRNEC
SEQ ID NO: 15
(GLP-1-TVAAPS-FGFR1c antibody heavy chain)
CATGGGGAGGGCACCTTCACCTCCGACGTCAGCTCTTACCTCGAGGGCCAAGCCGCCAAGGAGTTTATC
GCCTGGCTCGTGAAGGGGAGGACAGTCGCGGCGCCCAGCCAGGTGCAGCTGGTGCAGAGCGGCGCTGAG
GTGAAGAAGCCCGGCTCCTCCGTCAAGGTCAGCTGCAAGGCGAGCGGGCAGACATTCACCGGATACTAC
ATGCATTGGGTGCGCCAGGCCCCGGGGCAGGGGCTCGAGTGGATGGGGAGAATCATCCCCATCCTGGGC
ATCGCTCAGAAGTTCCAGGGACGCGTGACCATCACCGCCGACAAATCCACCAGCACCGCCTACATGGAA
CTGAGCTCCCTGCGCTCCGAGGACACCGCCGTGTATTATTGCGCCCGCGGGGGCGACCTGGGCGGCATG
GACGTGTGGGGCCAGGGCACACTAGTCACCGTCTCCTCAGCCAAAACAACAGCCCCATCGGTCTATCCA
CTGGCCCCTGTGTGTGGAGATACAACTGGCTCCTCGGTGACTCTAGGATGCCTGGTCAAGGGTTATTTC
CCTGAGCCAGTGACCTTGACCTGGAACTCTGGATCCCTGTCCAGTGGTGTGCACACCTTCCCAGCTGTC
CTGCAGTCTGACCTCTACACCCTCAGCAGCTCAGTGACTGTAACCTCGAGCACCTGGCCCAGCCAGTCC
ATCACCTGCAATGTGGCCCACCCGGCAAGCAGCACCAAGGTGGACAAGAAAATTGAGCCCAGAGGGCCC
ACAATCAAGCCCTGTCCTCCATGCAAATGCCCAGCACCTAACCTCGCGGGTGCACCATCCGTCTTCATC
TTCCCTCCAAAGATCAAGGATGTACTCATGATCTCCCTGAGCCCCATAGTCACATGTGTGGTGGTGGAT
GTGAGCGAGGATGACCCAGATGTCCAGATCAGCTGGTTTGTGAACAACGTGGAAGTACACACAGCTCAG
ACACAAACCCATAGAGAGGATTACAACAGTACTCTCCGGGTGGTCAGTGCCCTCCCCATCCAGCACCAG
GACTGGATGAGTGGCAAGGAGTTCAAATGCAAGGTCAACAACAAAGACCTCCCAGCGCCCATCGAGAGA
ACCATCTCAAAACCCAAAGGGTCAGTAAGAGCTCCACAGGTATATGTCTTGCCTCCACCAGAAGAAGAG
ATGACTAAGAAACAGGTCACTCTGACCTGCATGGTCACAGACTTCATGCCTGAAGACATTTACGTGGAG
TGGACCAACAACGGGAAAACAGAGCTAAACTACAAGAACACTGAACCAGTCCTGGACTCTGATGGTTCT
TACTTCATGTACAGCAAGCTGAGAGTGGAAAAGAAGAACTGGGTGGAAAGAAATAGCTACTCCTGTTCA
GTGGTCCACGAGGGTCTGCACAATCACCACACGACTAAGAGCTTCTCCCGGACTCCGGGTAAA
SEQ ID NO: 16
(GLP-1-TVAAPS-FGFR1c antibody heavy chain)
HGEGTFTSDVSSYLEGQAAKEFIAWLVKGRTVAAPSQVQLVQSGAEVKKPGSSVKVSCKASGQTFTGYY
MHWVRQAPGQGLEWMGRIIPILGIAQKFQGRVTITADKSTSTAYMELSSLRSEDTAVYYCARGGDLGGM
DVWGQGTLVTVSSAKTTAPSVYPLAPVCGDTTGSSVTLGCLVKGYFPEPVTLTWNSGSLSSGVHTFPAV
LQSDLYTLSSSVTVTSSTWPSQSITCNVAHPASSTKVDKKIEPRGPTIKPCPPCKCPAPNLAGAPSVFI
FPPKIKDVLMISLSPIVTCVVVDVSEDDPDVQISWFVNNVEVHTAQTQTHREDYNSTLRVVSALPIQHQ
DWMSGKEFKCKVNNKDLPAPIERTISKPKGSVRAPQVYVLPPPEEEMTKKQVTLTCMVTDFMPEDIYVE
WTNNGKTELNYKNTEPVLDSDGSYFMYSKLRVEKKNWVERNSYSCSVVHEGLHNHHTTKSFSRTPGK
SEQ ID NO: 17
(GLP-1-TVAAPS-FGFR1c antibody light chain)
CATGGGGAGGGCACCTTCACCTCCGACGTCAGCTCTTACCTCGAGGGCCAAGCCGCCAAGGAGTTTATC
GCCTGGCTCGTGAAGGGGAGGACAGTCGCGGCGCCCAGCGAGATCGTGCTGACCCAGAGCCCCCTCTCG
CTGCCCGTGACCCCCGGGGAGCCCGCCAGCATCTCCTGCCGCAGTAGCCAGAGCCTGAGGCATTCCAAT
GGCTACAACTACCTGGACTGGTACCTGCAGAAACCCGGCCAGAGCCCCCAGCTGCTCATCTACCTGGCG
AGTAACCGCGCCAGCGGGGTGCCCGACCGCTTCAGCGGCTCCGGCAGTGGAACCGACTTCACCCTGAAG
ATCTCCCGCGTGGAGGCGGAGGACGTGGGGGTGTATTACTGTATGCAGGCCCTCCAGATCCCCCCCACG
TTCGGCCCCGGCACCAAGGTGGACATCAAACGCACCGTCGCCGCCCCGACCGTGAGCATTTTCCCTCCC
AGCTCCGAGCAGCTGACGTCCGGCGGCGCCTCTGTGGTGTGCTTCCTCAACAACTTCTACCCCAAGGAC
ATCAACGTGAAGTGGAAGATCGACGGCTCCGAGAGACAGAACGGCGTGCTGAACAGCTGGACCGACCAG
GACAGCAAGGACTCCACCTACAGTATGAGCTCCACCCTGACCCTGACCAAGGACGAGTACGAGAGGCAT
AACTCTTATACCTGCGAGGCGACCCATAAGACCAGCACCTCCCCCATCGTCAAGAGCTTCAACCGCAAC
GAATGC
SEQ ID NO: 18
(GLP-1-TVAAPS-FGFR1c antibody light chain)
HGEGTFTSDVSSYLEGQAAKEFIAWLVKGRTVAAPSEIVLTQSPLSLPVTPGEPASISCRSSQSLRHSN
GYNYLDWYLQKPGQSPQLLIYLASNRASGVPDRFSGSGSGTDFTLKISRVEAEDVGVYYCMQALQIPPT
FGPGTKVDIKRTVAAPTVSIFPPSSEQLTSGGASVVCFLNNFYPKDINVKWKIDGSERQNGVLNSWTDQ
DSKDSTYSMSSTLTLTKDEYERHNSYTCEATHKTSTSPIVKSFNRNEC
SEQ ID NO: 19
(Exendin 4-G4S2-Scrambled antibody heavy chain)
CATGGGGAGGGCACTTTCACTAGCGACCTGAGCAAGCAGATGGAAGAAGAGGCCGTGAGGCTGTTCATT
GAGTGGCTCAAGAACGGAGGCCCCTCCTCCGGCGCCCCCCCCCCTAGCGGCGGATCCGGAGGCGGGGGC
AGTGGCGGGGGAGGTAGCGGTCAGGTCCAATTAGTGCAATCTGGGTCTGAGTTGAAGAAGCCTGGGGCC
TCAGTGAAGGTTTCCTGCAAGGCCTCTGGATACACCTTCACTAACTATGGAATGAACTGGGTGCGACAG
GCCCCTGGACAAGGGCTCGAGTGGATGGGATGGATAAACACCAGAAATGGAAAGTCAACATATGTTGAT
GACTTCAAGGGGCGGTTTGTCTTCTCCTTGGACACCTCTGTCAGCACGGCATATCTACAGATCAGCAGC
CTAAAGGCTGACGACACTGCAGTGTATTACTGTGCGAGAGAAGGGAATATGGATGGTTACTTCCCTTTT
ACTTACTGGGGCCAGGGTACACTAGTCACCGTCTCCTCAGCCAAAACAACAGCCCCATCGGTCTATCCA
CTGGCCCCTGTGTGTGGAGATACAACTGGCTCCTCGGTGACTCTAGGATGCCTGGTCAAGGGTTATTTC
CCTGAGCCAGTGACCTTGACCTGGAACTCTGGCTCCCTGTCCAGTGGTGTGCACACCTTCCCAGCTGTC
CTGCAGTCTGACCTCTACACCCTCAGCAGCTCAGTGACTGTAACCTCGAGCACCTGGCCCAGCCAGTCC
ATCACCTGCAATGTGGCCCACCCGGCAAGCAGCACCAAGGTGGACAAGAAAATTGAGCCCAGAGGGCCC
ACAATCAAGCCCTGTCCTCCATGCAAATGCCCAGCACCTAACCTCGCGGGTGCACCATCCGTCTTCATC
TTCCCTCCAAAGATCAAGGATGTACTCATGATCTCCCTGAGCCCCATAGTCACATGTGTGGTGGTGGAT
GTGAGCGAGGATGACCCAGATGTCCAGATCAGCTGGTTTGTGAACAACGTGGAAGTACACACAGCTCAG
ACACAAACCCATAGAGAGGATTACAACAGTACTCTCCGGGTGGTCAGTGCCCTCCCCATCCAGCACCAG
GACTGGATGAGTGGCAAGGAGTTCAAATGCAAGGTCAACAACAAAGACCTCCCAGCGCCCATCGAGAGA
ACCATCTCAAAACCCAAAGGGTCAGTAAGAGCTCCACAGGTATATGTCTTGCCTCCACCAGAAGAAGAG
ATGACTAAGAAACAGGTCACTCTGACCTGCATGGTCACAGACTTCATGCCTGAAGACATTTACGTGGAG
TGGACCAACAACGGGAAAACAGAGCTAAACTACAAGAACACTGAACCAGTCCTGGACTCTGATGGTTCT
TACTTCATGTACAGCAAGCTGAGAGTGGAAAAGAAGAACTGGGTGGAAAGAAATAGCTACTCCTGTTCA
GTGGTCCACGAGGGTCTGCACAATCACCACACGACTAAGAGCTTCTCCCGGACTCCGGGTAAA
SEQ ID NO: 20
(Exendin 4-G4S2-Scrambled antibody heavy chain)
HGEGTFTSDLSKQMEEEAVRLFIEWLKNGGPSSGAPPPSGGSGGGGSGGGGSGQVQLVQSGSELKKPGA
SVKVSCKASGYTFTNYGMNWVRQAPGQGLEWMGWINTRNGKSTYVDDFKGRFVFSLDTSVSTAYLQISS
LKADDTAVYYCAREGNMDGYFPFTYWGQGTLVTVSSAKTTAPSVYPLAPVCGDTTGSSVTLGCLVKGYF
PEPVTLTWNSGSLSSGVHTFPAVLQSDLYTLSSSVTVTSSTWPSQSITCNVAHPASSTKVDKKIEPRGP
TIKPCPPCKCPAPNLAGAPSVFIFPPKIKDVLMISLSPIVTCVVVDVSEDDPDVQISWFVNNVEVHTAQ
TQTHREDYNSTLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEKKNWVERNSYSCS
VVHEGLHNHHTTKSFSRTPGK
SEQ ID NO: 21
(Exendin 4-G4S2-Scrambled antibody light chain)
CATGGGGAGGGCACTTTCACTAGCGACCTGAGCAAGCAGATGGAAGAAGAGGCCGTGAGGCTGTTCATT
GAGTGGCTCAAGAACGGAGGCCCCTCCTCCGGCGCCCCCCCCCCTAGCGGCGGATCCGGAGGCGGGGGC
AGTGGCGGGGGAGGTAGCGGTGATATTGTCATGACTCAGTCTCCATCATCCCTGTCCGCATCAGTAGGA
GACAGGGTCACCATCACCTGCAAGGCTTCTCAGAATGTGGGTACTAATGTAGCCTGGTATCAACAGAAA
CCAGGGAAAGCTCCTAAAGCACTGATTTACTCGGCATCCTATCGGTACAGTGGAGTCCCTGATCGCTTC
TCAGGCAGTGGCTCCGGGACAGATTTCACTCTCACCATCAGCAGTCTGCAGCCTGAAGACTTCGCAACG
TATTACTGTCAGCAATATAACAGCTATCCTCTCACGTTCGGTGGTGGTACCAAGGTGGAAATAAAACGT
ACGGTCGCCGCCCCGACCGTGAGCATTTTCCCTCCCAGCTCCGAGCAGCTGACGTCCGGCGGCGCCTCT
GTGGTGTGCTTCCTCAACAACTTCTACCCCAAGGACATCAACGTGAAGTGGAAGATCGACGGCTCCGAG
AGACAGAACGGCGTGCTGAACAGCTGGACCGACCAGGACAGCAAGGACTCCACCTACAGTATGAGCTCC
ACCCTGACCCTGACCAAGGACGAGTACGAGAGGCATAACTCTTATACCTGCGAGGCGACCCATAAGACC
AGCACCTCCCCCATCGTCAAGAGCTTCAACCGCAACGAATGC
SEQ ID NO: 22
(Exendin 4-G4S2-Scrambled antibody light chain)
HGEGTFTSDLSKQMEEEAVRLFIEWLKNGGPSSGAPPPSGGSGGGGSGGGGSGDIVMTQSPSSLSASVG
DRVTITCKASQNVGTNVAWYQQKPGKAPKALIYSASYRYSGVPDRFSGSGSGTDFTLTISSLQPEDFAT
YYCQQYNSYPLTFGGGTKVEIKRTVAAPTVSIFPPSSEQLTSGGASVVCFLNNFYPKDINVKWKIDGSE
RQNGVLNSWTDQDSKDSTYSMSSTLTLTKDEYERHNSYTCEATHKTSTSPIVKSFNRNEC
SEQ ID NO: 23
(Alternative Scrambled antibody light chain)
GATATTGTCATGACTCAGTCTCCATCATCCCTGTCCGCATCAGTAGGAGACAGGGTCACCATCACCTGC
AAGGCTTCTCAGAATGTGGGTACTAATGTAGCCTGGTATCAACAGAAACCAGGGAAAGCTCCTAAAGCA
CTGATTTACTCGGCATCCTATCGGTACAGTGGAGTCCCTGATCGCTTCTCAGGCAGTGGATCCGGGACA
GATTTCACTCTCACCATCAGCAGTCTGCAGCCTGAAGACTTCGCAACGTATTACTGTCAGCAATATAAC
AGCTATCCTCTCACGTTCGGTGGTGGTACCAAGGTGGAAATAAAACGTACGGTGGCCGCCCCGACCGTG
AGCATTTTCCCTCCCAGCTCCGAGCAGCTGACGTCCGGCGGCGCCTCTGTGGTGTGCTTCCTCAACAAC
TTCTACCCCAAGGACATCAACGTGAAGTGGAAGATCGACGGCTCCGAGAGACAGAACGGCGTGCTGAAC
AGCTGGACCGACCAGGACAGCAAGGACTCCACCTACAGTATGAGCTCCACCCTGACCCTGACCAAGGAC
GAGTACGAGAGGCATAACTCTTATACCTGCGAGGCGACCCATAAGACCAGCACCTCCCCCATCGTCAAG
AGCTTCAACCGCAACGAATGC
SEQ ID NO: 24
(Alternative Scrambled antibody light chain)
DIVMTQSPSSLSASVGDRVTITCKASQNVGTNVAWYQQKPGKAPKALIYSASYRYSGVPDRFSGSGSGT
DFTLTISSLQPEDFATYYCQQYNSYPLTFGGGTKVEIKRTVAAPTVSIFPPSSEQLTSGGASVVCFLNN
FYPKDINVKWKIDGSERQNGVLNSWTDQDSKDSTYSMSSTLTLTKDEYERHNSYTCEATHKTSTSPIVK
SFNRNEC
SEQ ID NO: 25
(GLP-1- G4S2-Scr (scrambled) Antibody heavy chain)
CATGGGGAGGGCACCTTCACCTCCGACGTCAGCTCTTACCTCGAGGGCCAAGCCGCCAAGGAGTTTATC
GCCTGGCTCGTGAAGGGGAGGGGATCCGGAGGCGGGGGCAGTGGCGGGGGAGGTAGCGGTCAGGTCCAA
TTAGTGCAATCTGGGTCTGAGTTGAAGAAGCCTGGGGCCTCAGTGAAGGTTTCCTGCAAGGCCTCTGGA
TACACCTTCACTAACTATGGAATGAACTGGGTGCGACAGGCCCCTGGACAAGGGCTCGAGTGGATGGGA
TGGATAAACACCAGAAATGGAAAGTCAACATATGTTGATGACTTCAAGGGGCGGTTTGTCTTCTCCTTG
GACACCTCTGTCAGCACGGCATATCTACAGATCAGCAGCCTAAAGGCTGACGACACTGCAGTGTATTAC
TGTGCGAGAGAAGGGAATATGGATGGTTACTTCCCTTTTACTTACTGGGGCCAGGGTACACTAGTCACC
GTCTCCTCAGCCAAAACAACAGCCCCATCGGTCTATCCACTGGCCCCTGTGTGTGGAGATACAACTGGC
TCCTCGGTGACTCTAGGATGCCTGGTCAAGGGTTATTTCCCTGAGCCAGTGACCTTGACCTGGAACTCT
GGCTCCCTGTCCAGTGGTGTGCACACCTTCCCAGCTGTCCTGCAGTCTGACCTCTACACCCTCAGCAGC
TCAGTGACTGTAACCTCGAGCACCTGGCCCAGCCAGTCCATCACCTGCAATGTGGCCCACCCGGCAAGC
AGCACCAAGGTGGACAAGAAAATTGAGCCCAGAGGGCCCACAATCAAGCCCTGTCCTCCATGCAAATGC
CCAGCACCTAACCTCGCGGGTGCACCATCCGTCTTCATCTTCCCTCCAAAGATCAAGGATGTACTCATG
ATCTCCCTGAGCCCCATAGTCACATGTGTGGTGGTGGATGTGAGCGAGGATGACCCAGATGTCCAGATC
AGCTGGTTTGTGAACAACGTGGAAGTACACACAGCTCAGACACAAACCCATAGAGAGGATTACAACAGT
ACTCTCCGGGTGGTCAGTGCCCTCCCCATCCAGCACCAGGACTGGATGAGTGGCAAGGAGTTCAAATGC
AAGGTCAACAACAAAGACCTCCCAGCGCCCATCGAGAGAACCATCTCAAAACCCAAAGGGTCAGTAAGA
GCTCCACAGGTATATGTCTTGCCTCCACCAGAAGAAGAGATGACTAAGAAACAGGTCACTCTGACCTGC
ATGGTCACAGACTTCATGCCTGAAGACATTTACGTGGAGTGGACCAACAACGGGAAAACAGAGCTAAAC
TACAAGAACACTGAACCAGTCCTGGACTCTGATGGTTCTTACTTCATGTACAGCAAGCTGAGAGTGGAA
AAGAAGAACTGGGTGGAAAGAAATAGCTACTCCTGTTCAGTGGTCCACGAGGGTCTGCACAATCACCAC
ACGACTAAGAGCTTCTCCCGGACTCCGGGTAAA
SEQ ID NO: 26
(GLP-1- G4S2-Scr (scrambled) antibody heavy chain)
HGEGTFTSDVSSYLEGQAAKEFIAWLVKGRGSGGGGSGGGGSGQVQLVQSGSELKKPGASVKVSCKASG
YTFTNYGMNWVRQAPGQGLEWMGWINTRNGKSTYVDDFKGRFVFSLDTSVSTAYLQISSLKADDTAVYY
CAREGNMDGYFPFTYWGQGTLVTVSSAKTTAPSVYPLAPVCGDTTGSSVTLGCLVKGYFPEPVTLTWNS
GSLSSGVHTFPAVLQSDLYTLSSSVTVTSSTWPSQSITCNVAHPASSTKVDKKIEPRGPTIKPCPPCKC
PAPNLAGAPSVFIFPPKIKDVLMISLSPIVTCVVVDVSEDDPDVQISWFVNNVEVHTAQTQTHREDYNS
TLRVVSALPIQHQDWMSGKEFKCKVNNKDLPAPIERTISKPKGSVRAPQVYVLPPPEEEMTKKQVTLTC
MVTDFMPEDIYVEWTNNGKTELNYKNTEPVLDSDGSYFMYSKLRVEKKNWVERNSYSCSVVHEGLHNHH
TTKSFSRTPGK
SEQ ID NO: 27
(GLP-1- G4S2-Scr (scrambled) antibody light chain)
CATGGGGAGGGCACCTTCACCTCCGACGTCAGCTCTTACCTCGAGGGCCAAGCCGCCAAGGAGTTTATC
GCCTGGCTCGTGAAGGGGAGGGGATCCGGAGGCGGGGGCAGTGGCGGGGGAGGTAGCGGTGATATTGTC
ATGACTCAGTCTCCATCATCCCTGTCCGCATCAGTAGGAGACAGGGTCACCATCACCTGCAAGGCTTCT
CAGAATGTGGGTACTAATGTAGCCTGGTATCAACAGAAACCAGGGAAAGCTCCTAAAGCACTGATTTAC
TCGGCATCCTATCGGTACAGTGGAGTCCCTGATCGCTTCTCAGGCAGTGGCTCCGGGACAGATTTCACT
CTCACCATCAGCAGTCTGCAGCCTGAAGACTTCGCAACGTATTACTGTCAGCAATATAACAGCTATCCT
CTCACGTTCGGTGGTGGTACCAAGGTGGAAATAAAACGTACGGTCGCCGCCCCGACCGTGAGCATTTTC
CCTCCCAGCTCCGAGCAGCTGACGTCCGGCGGCGCCTCTGTGGTGTGCTTCCTCAACAACTTCTACCCC
AAGGACATCAACGTGAAGTGGAAGATCGACGGCTCCGAGAGACAGAACGGCGTGCTGAACAGCTGGACC
GACCAGGACAGCAAGGACTCCACCTACAGTATGAGCTCCACCCTGACCCTGACCAAGGACGAGTACGAG
AGGCATAACTCTTATACCTGCGAGGCGACCCATAAGACCAGCACCTCCCCCATCGTCAAGAGCTTCAAC
CGCAACGAATGC
SEQ ID NO: 28
GLP-1- G4S2-SCR (SCRAMBLED) ANTIBODY LIGHT CHAIN
HGEGTFTSDVSSYLEGQAAKEFIAWLVKGRGSGGGGSGGGGSGDIVMTQSPSSLSASVGDRVTITCKAS
QNVGTNVAWYQQKPGKAPKALIYSASYRYSGVPDRFSGSGSGTDFTLTISSLQPEDFATYYCQQYNSYP
LTFGGGTKVEIKRTVAAPTVSIFPPSSEQLTSGGASVVCFLNNFYPKDINVKWKIDGSERQNGVLNSWT
DQDSKDSTYSMSSTLTLTKDEYERHNSYTCEATHKTSTSPIVKSFNRNEC
SEQ ID NO: 29
(FGFR1c antibody VH)
CAGGTGCAGCTGGTGCAGAGCGGCGCTGAGGTGAAGAAGCCCGGCTCCTCCGTCAAGGTCAGCTGCAAG
GCGAGCGGGCAGACATTCACCGGATACTACATGCATTGGGTGCGCCAGGCCCCGGGGCAGGGGCTCGAG
TGGATGGGGAGAATCATCCCCATCCTGGGCATCGCTCAGAAGTTCCAGGGACGCGTGACCATCACCGCC
GACAAATCCACCAGCACCGCCTACATGGAACTGAGCTCCCTGCGCTCCGAGGACACCGCCGTGTATTAT
TGCGCCCGCGGGGGCGACCTGGGCGGCATGGACGTGTGGGGCCAGGGC
SEQ ID NO: 30
(FGFR1c antibody VH)
QVQLVQSGAEVKKPGSSVKVSCKASGQTFTGYYMHWVRQAPGQGLEWMGRIIPILGIAQKFQGRVTITA
DKSTSTAYMELSSLRSEDTAVYYCARGGDLGGMDVWGQG
SEQ ID NO: 31
(FGFR1c antibody VL)
GAGATCGTGCTGACCCAGAGCCCCCTCTCGCTGCCCGTGACCCCCGGGGAGCCCGCCAGCATCTCCTGC
CGCAGTAGCCAGAGCCTGAGGCATTCCAATGGCTACAACTACCTGGACTGGTACCTGCAGAAACCCGGC
CAGAGCCCCCAGCTGCTCATCTACCTGGCGAGTAACCGCGCCAGCGGGGTGCCCGACCGCTTCAGCGGC
TCCGGCAGTGGAACCGACTTCACCCTGAAGATCTCCCGCGTGGAGGCGGAGGACGTGGGGGTGTATTAC
TGTATGCAGGCCCTCCAGATCCCCCCCACGTTCGGCCCCGGCACCAAGGTGGACATCAAACGCACCGTC
GCCGCC
SEQ ID NO: 32
(FGFR1c antibody VL)
EIVLTQSPLSLPVTPGEPASISCRSSQSLRHSNGYNYLDWYLQKPGQSPQLLIYLASNRASGVPDRFSG
SGSGTDFTLKISRVEAEDVGVYYCMQALQIPPTFGPGTKVDIKRTVAA
SEQ ID NO: 33
(Mammalian signal sequence)
MGWSCIILFLVATATGVHS
SEQ ID NO: 34
LINKER G4S4
GSGGGGSGGGGSGGGGSGGGGSG
SEQ ID NO: 35
LINKER TVAAPS
TVAAPS
SEQ ID NO: 36
LINKER ASTK
ASTKGPS
SEQ ID NO: 37
LINKER G4S
GSSSS

### BRIEF DESCRIPTION OF FIGURES

FIG. 1 shows the binding of FGFR1cA1, Ex4FGFR1A1cH, Ex4FGFR1cA1H/L, and Ex4FGFR1cA1L to FGFR1c.

FIG. 2 shows the binding of EX4G4S4FGFR1cH, EX4G4S4FGFR1cL, EX4ASTKFGFR1cH, EX4ASTKFGFR1cL, Ex4 FGFR1cA1H, and FGFR1cA1 to FGFR1c.

FIG. 3 shows the binding of EX4TVAAPSFGFR1cL, GLP1TVAAPSFGFR1cL, EX4TVAAPSFGFR1cH, GLP1TVAAPSFGFR1cH, G4S2FGFR1cL and G4S2FGFR1cH to FGFR1c.

FIG. 4 shows the inhibition of FGFR1c binding to its ligand FGF in the presence of FGFR1cA1, Ex4FGFR1cA1 H, Ex4FGFR1cA1 L, Ex4FGFR1cA1 H/L and FGFR1 b antibody.

FIG. 5 shows the effects on diet consumption in mice after administration of the compositions and dual targeting proteins of the invention.

FIG. 6 shows the effects on weight loss in mice after administration of the compositions and dual targeting proteins of the invention.

FIG. 7 shows the effects on % reduction in fat/lean tissue in mice after administration of the compositions and dual targeting proteins of the invention.

FIG. 8 shows schematics of some embodiments of the dual targeting proteins of the invention.

FIG. 9 shows diet consumption in mice after frequent dosing of the compositions and dual targeting proteins of the invention.

FIG. 10 shows shows the effects on weight loss in mice after frequent dosing of the compositions and dual targeting proteins of the invention.

FIG. 11 shows the effects on % reduction in fat/lean tissue in mice after frequent dosing of the compositions and dual targeting proteins of the invention.

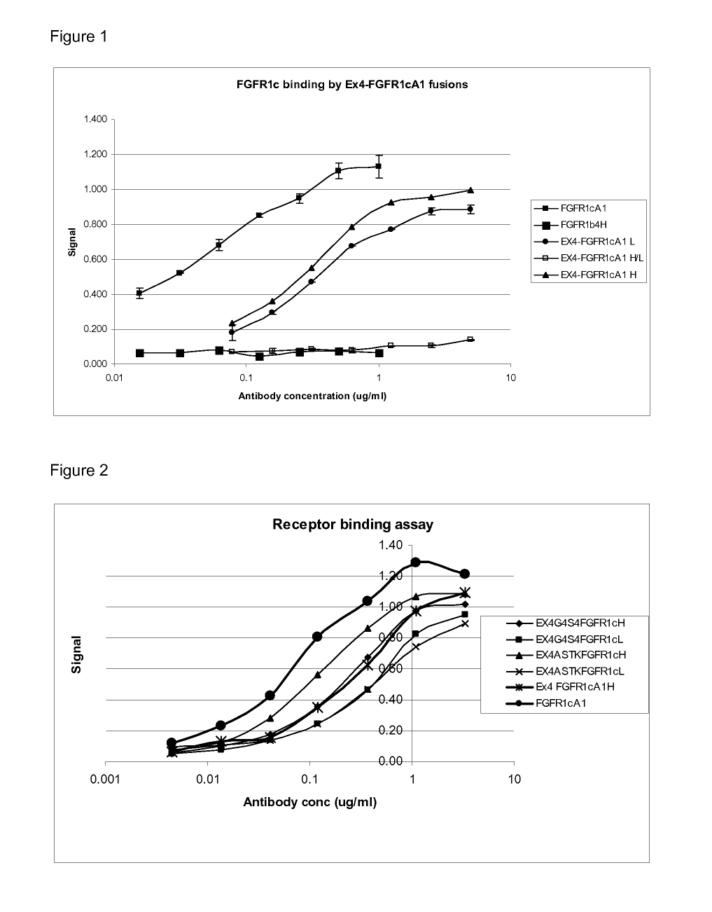

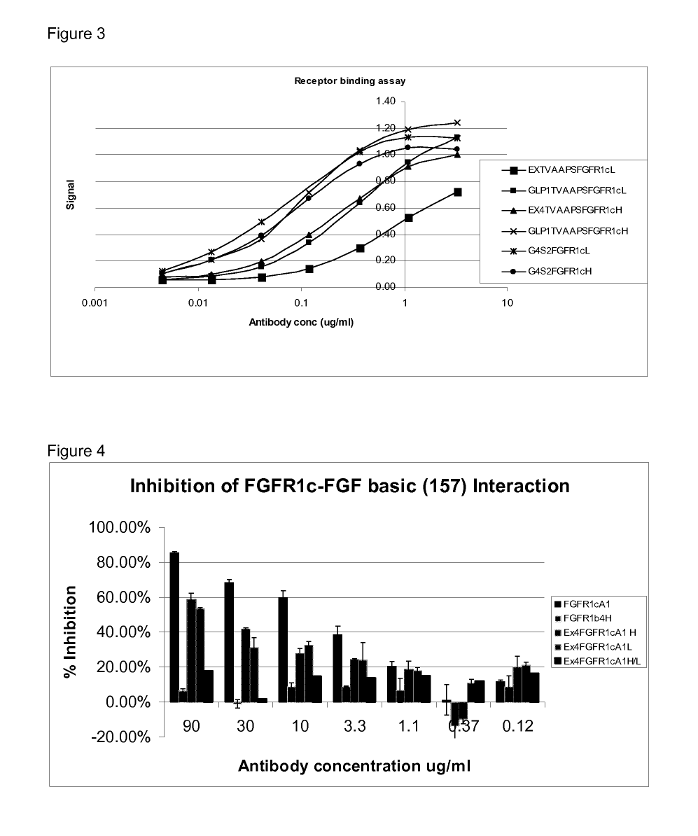

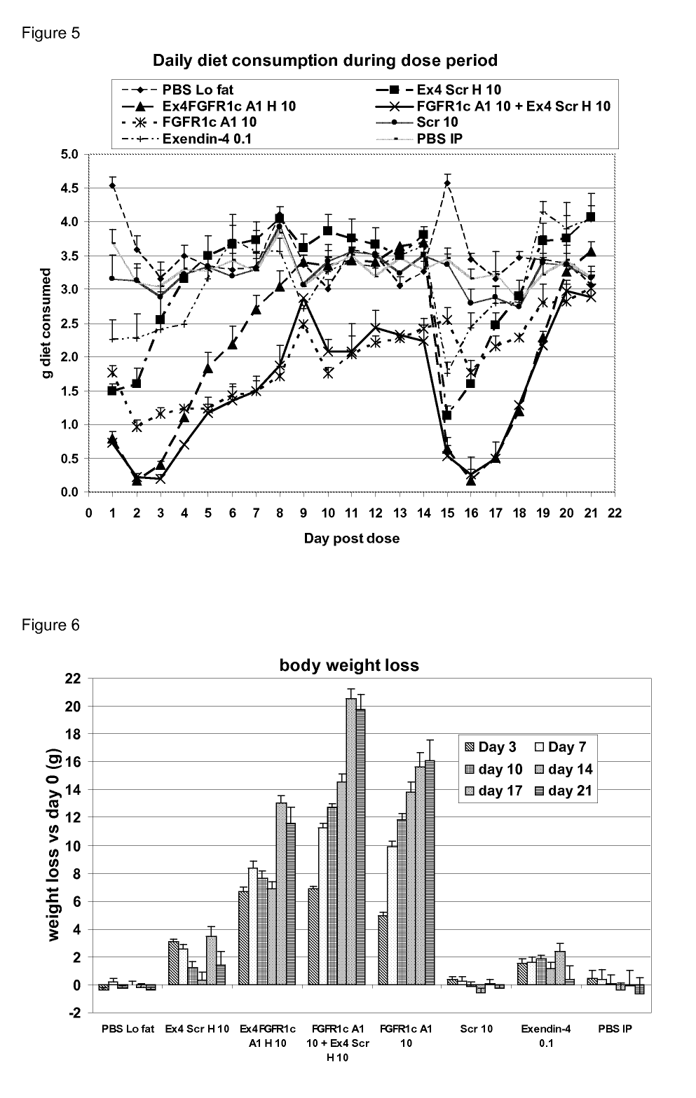

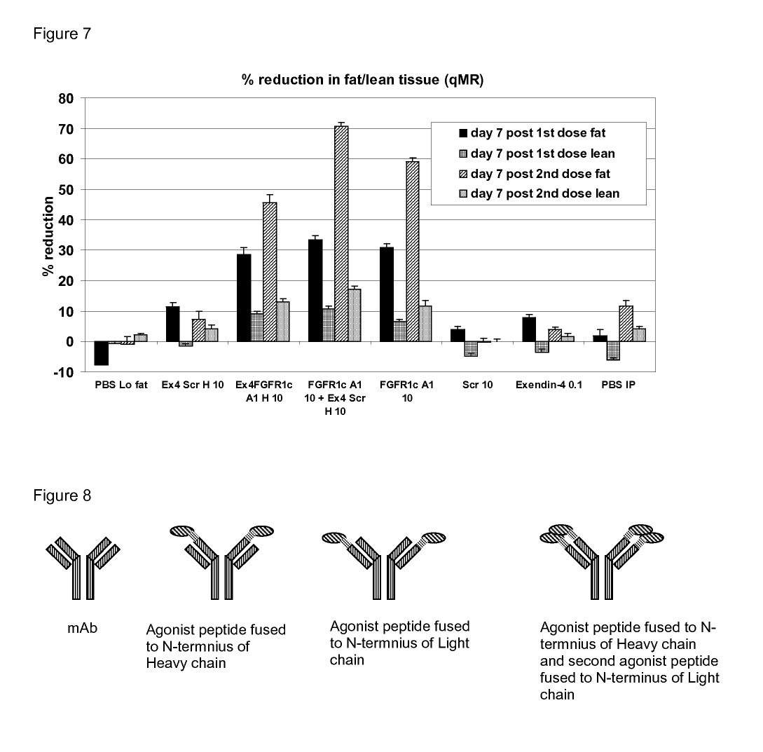

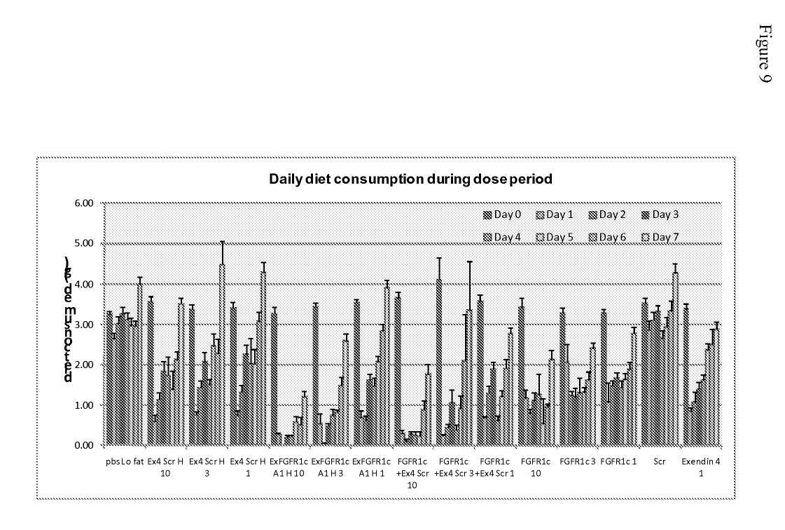

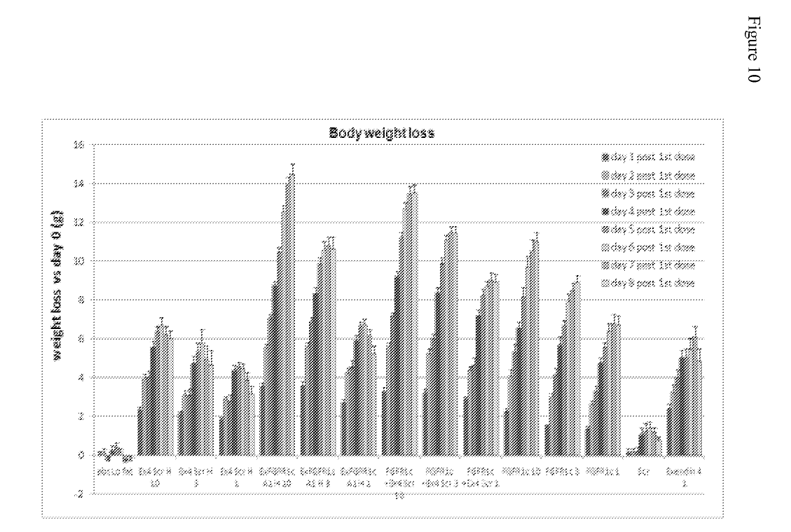

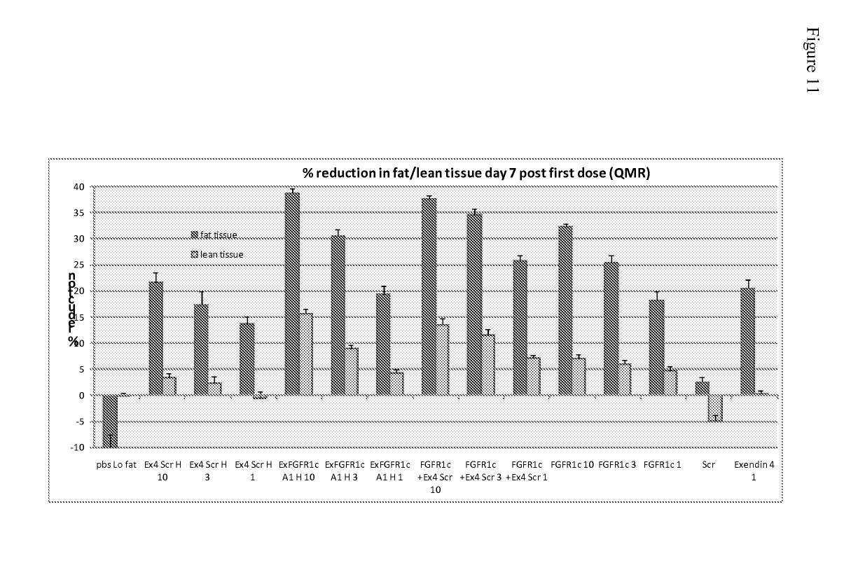

1-33. (canceled)

34. A composition comprising an FGFR1c antagonist and an agonist peptide.

35. A dual targeting protein comprising an antigen binding protein which is capable of binding to FGFR1c, and which is linked to one or more agonist peptides.

36. The composition of claim 35 or the dual targeting protein of claim 35 wherein the antigen binding protein is an anti-FGFR1c antibody or an antigen binding fragment thereof.

37. The composition or dual targeting protein of claim 35 wherein the antigen binding protein comprises a dAb.

38. The composition or dual targeting protein of claim 37 wherein the antigen binding protein is an antibody

39. The composition or dual targeting protein of claim 34, wherein said agonist peptide is a GLP-1 agonist.

40. The composition or dual targeting protein of claim 39, wherein said GLP-1 agonist is selected from the group consisting of: human GLP-1, exendin 3 and exendin 4 or a fragment or variant thereof

41. A composition or dual targeting protein according to claim 35 wherein the antigen binding protein comprises the CDRs contained in the VH region set out in SEQ ID NO:30 and CDRs contained in the VL region set out in SEQ ID NO:32.

42. A pharmaceutical composition comprising a dual targeting protein of claim 35 and a pharmaceutically acceptable carrier.

43. A composition or dual targeting protein according to claim 34 for use in medicine.

44. The use of a composition or dual targeting protein according to claim 34 in the treatment of obesity.

45. The use of a composition or dual targeting protein according to claim 34 in the reduction of body weight.

46. The use of a composition or dual targeting protein according to claims 34 for reducing food intake in a patient.

47. The use of a composition or dual targeting protein according to claim 34 for inhibiting gastric emptying in a patient.
